(19) United States
(12) Patent Application Publication
(10) Pub. No.: US 2016/0365198 A1
(43) Pub. Date: Dec. 15, 2016

(54) LIQUID COLUMN-BASED CAPACITIVE SENSORS

(71) Applicant: THE REGENTS OF THE UNIVERSITY OF CALIFORNIA, Oakland, CA (US)

(72) Inventors: Tingrui Pan, Woodland, CA (US); Baoqing Nie, Davis, CA (US); Ruya Li, Davis, CA (US); James D. Brandt, Los Angeles, CA (US)

(73) Assignee: THE REGENTS OF THE UNIVERSITY OF CALIFORNIA, Oakland, CA (US)

(21) Appl. No.: 15/178,092

(22) Filed: Jun. 9, 2016

Related U.S. Application Data
(63) Continuation of application No. PCT/US2014/070187, filed on Dec. 13, 2014.
(60) Provisional application No. 61/916,196, filed on Dec. 14, 2013.

(51) Int. Cl. H01G 11/08; A61B 5/021; G01L 1/14

(52) U.S. Cl. CPC H01G 11/08; G01L 1/142; A61B 5/021; A61B 2562/0247

(57) ABSTRACT

A liquid column-based normal/shear pressure/force sensing device having an elastic electrolyte-electrode contact with large interfacial capacitance to achieve high sensitivity and resolution with flexible and transparent constructs.

### CROSS-REFERENCE TO RELATED APPLICATIONS

This application is a 35 U.S.C. §111(a) continuation of PCT international application number PCT/US2014/070187 filed on Dec. 14, 2014, incorporated herein by reference in its entirety, which claims priority to, and the benefit of, U.S. provisional patent application Ser. No. 61/916,196 filed on Dec. 14, 2013, incorporated herein by reference in its entirety. Priority is claimed to each of the foregoing applications.

The above-referenced PCT international application was published as PCT International Publication No. WO 2015/089491 on Jun. 18, 2015, which publication is incorporated herein by reference in its entirety.

### STATEMENT REGARDING FEDERALLY SPONSORED RESEARCH OR DEVELOPMENT

This invention was made with Government support under ECCS-0846502 and ECCS-1307831, awarded by the National Science Foundation. The Government has certain rights in the invention.

### INCORPORATION-BY-REFERENCE OF COMPUTER PROGRAM APPENDIX

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND

1. Technical Field

This technology pertains generally to sensing devices, and more particularly to a droplet-based capacitive pressure sensing device.

2. Background Discussion

Microfluidic-based sensors have been an active area of research for their excellent flexibility, high sensitivity, simple fabrication, and wide adaptability. A variety of sensing and actuation mechanisms have been incorporated in the development of microfluidic sensing devices, the majority of which rely on sensing changes in a physical property (e.g., optical, electrical or mechanical) induced by fluidic displacement, and/or new material functionality introduced to working fluids (e.g., as optical and electromagnetic waveguides).

However, the existing microfluidic sensors suffer from one or more shortcomings, such as being influenced by environmental effects, and/or insufficient pressure sensitivity and resolution.

### BRIEF SUMMARY

In general terms, the description herein pertains to novel liquid column-based normal/shear pressure/force sensing devices that provide ultrahigh levels of pressure sensitivity and resolution, while overcoming numerous environmental sensitivity issues of prior microfluidic sensors. In one embodiment, a device according to the present description comprises an elastic electrolyte-electrode contact with large interfacial capacitance to achieve high sensitivity and resolution with flexible and transparent constructs. In one embodiment, a capacitive sensor device according to the present description comprises conductive liquid columns sandwiched between two polymeric membranes coated with conductive materials, serving as the electrodes, forming an electrical double layer with remarkable unit-area capacitance. Under external loads, the membrane deformation results in expansion of the liquid-electrode contact, which offers a completely new capacitive sensing scheme with significant increase in the sensitivity.

Another aspect is an iontronic tactile sensing array, referred to as iontronic microdroplet array (IMA), using the novel droplet-enabled interfacial capacitive sensing principle. As an emerging alternative to the existing solid-state capacitive sensors, the IMA utilizes a highly capacitive EDL interface upon the electrode-electrolyte contact as the sensing element to achieve ultrahigh mechanical-to-electrical sensitivity (of 0.43 nF kPa⁻¹) and fine pressure resolution (of 33 Pa) in a 3×3×0.2 mm³ packaging, in comparison with the highest reported sensitivity of 0.8 nF kPa⁻¹ with a much larger footprint (of 6×6 mm²).

The novel flexible sensors can be used for artificial skin applications, in which both the normal and shear force/pressure can be detected. Various embodiments of the description may exhibit one or more of the following characteristics:

(a) ultrahigh sensitivity and resolution;

(b) simple fabrication;

(c) mechanical flexibility and optical transparency;

(d) fast dynamic response;

(e) high repeatability; and

(f) immunity to environmental noises, e.g., stray capacitance.

Further aspects of the technology will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1A is a schematic diagram of a liquid-based impedance pressure sensor comprising a partially-filled liquid chamber in accordance with the present description.

FIG. 1B is a schematic diagram of the liquid-based impedance pressure sensor of FIG. 1A in a compressed configuration.

FIG. 2A is a schematic diagram of a liquid-based impedance pressure sensor comprising a wholly-filled liquid chamber in accordance with the present description.

FIG. 2B is a schematic diagram of the liquid-based impedance pressure sensor of FIG. 2A in a compressed configuration.

FIG. 3A is a schematic circuit diagram of a liquid-based impedance pressure sensor in a capacitive mode in accordance with the present description.

FIG. 3B is a schematic circuit diagram of a liquid-based impedance pressure sensor in a resistive mode in accordance with the present description.

FIG. 3C is a schematic circuit diagram of a liquid-based impedance pressure sensor in an impedance mode in accordance with the present description.

FIG. 4A shows a schematic top view of a liquid-based impedance pressure sensor having dual electrode layers.

FIG. 4B shows a schematic section view of the sensor of FIG. 4A.

FIG. 4C shows a second schematic section view of the sensor of FIG. 4A.

FIG. 5A shows a schematic top view of a liquid-based impedance pressure sensor having a single electrode layer.

FIG. 5B shows a schematic section view of the sensor of FIG. 5A.

FIG. 5C shows a second schematic section view of the sensor of FIG. 5A.

FIG. 6A shows a schematic top view of a liquid-based impedance pressure sensor having dual electrode layers, with one of the electrodes embedded in a cavity.

FIG. 6B shows a schematic section view of the sensor of FIG. 6A.

FIG. 6C shows a second schematic section view of the sensor of FIG. 6A.

FIG. 7A shows a schematic top view of a liquid-based impedance pressure sensor having an electrode layer and cavity configuration of FIG. 6A.

FIG. 7B shows a schematic section view of the sensor of FIG. 7A.

FIG. 7C shows a second schematic section view of the sensor of FIG. 7A.

FIG. 8A illustrates a plan section view of a liquid-based impedance pressure sensor comprising a chamber and a channel formed via top and bottom substrate layers and supporting layers.

FIG. 8B shows a schematic section view of the sensor of FIG. 8A.

FIG. 9 illustrates a plan section view of a liquid-based impedance pressure sensor comprising a chamber and a plurality of channels similar to that shown in FIG. 8A through FIG. 8B.

FIG. 10A shows a plan section view of a liquid-based impedance pressure sensor with liquid disposed on the right side of chamber.

FIG. 10B is an orthogonal section of the view of FIG. 10A.

FIG. 11A shows a plan section view of a liquid-based impedance pressure sensor with liquid formed as a droplet disposed within a location of the chamber.

FIG. 11B shows a schematic section view of the sensor of FIG. 11A.

FIG. 12 shows a side section view of a liquid-based impedance pressure sensor having a hydrophilic area within the chamber to form an anchor structure.

FIG. 13 shows a side section view of a liquid-based impedance pressure sensor having a micropillar structure within the chamber to form an anchor structure.

FIG. 14A shows a plan section view of a liquid-based impedance pressure sensor with a bump structure applied to the sensor for detection of both normal and shear force measurements.

FIG. 14B shows a schematic section view of the sensor of FIG. 14A.

FIG. 14C shows a second schematic section view of the sensor of FIG. 14A.

FIG. 15A shows a plan section view of a liquid-based impedance pressure sensor with a bump structure and multiple channels for detection of both normal force and two-dimensional shear force.

FIG. 15B shows a schematic section view of the sensor of FIG. 15A.

FIG. 16 is a cross-sectional plan view of co-planar electrodes to be used with a liquid-based impedance pressure sensor.

FIG. 17A is a cross-sectional plan view of co-planar electrodes disposed in an opposing spiral pattern.

FIG. 17B is a cross-sectional view of the electrodes of FIG. 17A.

FIG. 18 is a cross-sectional plan view of interdigitating electrodes to be used with a liquid-based impedance pressure sensor.

FIG. 19 is a cross-sectional plan view of a pair of spiral electrodes to be used with a liquid-based impedance pressure sensor.

FIG. 20A is plan section view of a liquid-based impedance pressure sensor having two electrode plates having the pattern of FIG. 17A and FIG. 17B.

FIG. 20B is a cross-sectional view of the sensor of FIG. 20A.

FIG. 21A is a cross-sectional plan view of a liquid-based impedance pressure sensor with a cantilevered cavity.

FIG. 21B is a cross-sectional view of the sensor of FIG. 21A.

FIG. 22A is a cross-sectional plan view of a liquid-based impedance pressure sensor with a cantilevered cavity and multiple liquid droplets.

FIG. 22B is a cross-sectional view of the sensor of FIG. 22A.

FIG. 23 shows a schematic diagram of an exemplary data acquisition system for the IMA devices of the present description.

FIG. 24 is a plot showing the frequency dependence of the interfacial EDL capacitance at the electronic-ionic interface. The lower marks indicate the measurement results from IL on the original ITO/PET surface, while the upper marks show those measured from IL on the hydrophobic-modified ITO/PET surface.

FIG. 25A is a plot showing spatial resolution (by varying the pixel sizes from 1 to 3 mm with a constant membrane thickness of 75 μm) of the device of the present description.

FIG. 25B is a plot showing the membrane thickness (by altering the thickness from 75 to 175 μm in a fixed pixel size of 2 mm) of the device of the present description, where the measurement results (dots) with the corresponding fitting curve (dash lines) are plotted against the theoretical predications (solid lines).

FIG. 26A through FIG. 26C are plots showing the time-resolved sensor response measurements under repetitive mechanical loads in the frequency of 1 Hz (FIG. 26A), 10 Hz (FIG. 26B), and 100 Hz (FIG. 26C), where the square curves indicate the input voltage to drive the electromagnetic pin actuator, and the other curves are the output capacitance measured from a single sensing unit of the IMA device.

FIG. 27A is a plot showing capacitive changes as a function of repetitive cycles of the external pressure (>20,000), suggesting the mechanical reliability and robustness of the IMA device.

FIG. 27B is a plot showing the influence on the temperature variations on the device of the present description. The frequency spectrums of the initial capacitances of sensing devices with the solid-liquid contact area of 4.2 mm² are measured under the temperature changing from 5° C. to 50° C., where the corresponding the capacitive changes is found to be less than 10%.

FIG. 28A illustrates a schematic diagram of a system for real-time wrist pulse measurements using a transparent IMA device comprising a 5×5 array, with each pixel size of 3×3 mm² embedded in a flexible wrist band for non-invasive pressure wave recording.

FIG. 28B is an expanded view of the array of FIG. 28A.

FIG. 29 is a diagram illustrating the spatial distributions of the pulse intensities mapped by the IMA matrix of FIG. 28A. The highest pressure variation readings, marked as I, II and III, are located above the radial artery, in corresponding to the positions of the three units in FIG. 28A.

FIG. 30A through FIG. 30C are plots showing time-resolved strongest pulses recorded by the marked sensing units in FIG. 29.

FIG. 31 is a close-up view of one pulse signal recorded by sensor II in FIG. 30B, in which P1, P2 and P3 represent the three consecutive peaks of the recorded pressure wave in each cardiovascular cycle.

### DETAILED DESCRIPTION

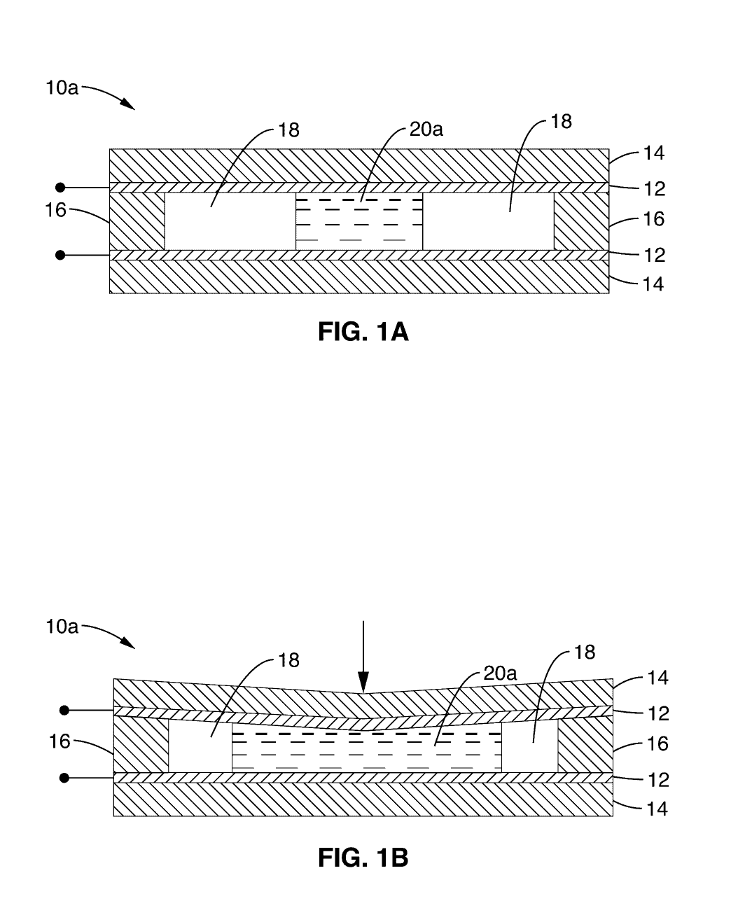

FIG. 1A and FIG. 1B show schematic diagrams of a liquid-based impedance pressure sensor 10a comprising a partially-filled liquid chamber 18 in accordance with the present description. The pressure/force sensor 10a utilizes an electrical double layer (EDL) of parallel electrode layers/plates 12 at the liquid/solid interface as the sensing elements. Electrodes 12 define opposing ends of chamber 18, which houses a droplet or column of liquid 20a such as an electrolyte solution. Chamber 18 is further bounded by supporting layer 16 of building material. Additional building material may be disposed in substrate/membrane layers 14. Layers 14 may comprise one or more of flexible membranes or rigid substrates, or a combination thereof.

Under external mechanical loads (FIG. 1B), one of more of the deformable membranes 14 will change shape, and as a result, the contact area of the liquid 20a-electrode 12 interface experiences expansion (assuming incompressible fluid with unaltered volume of the liquid).

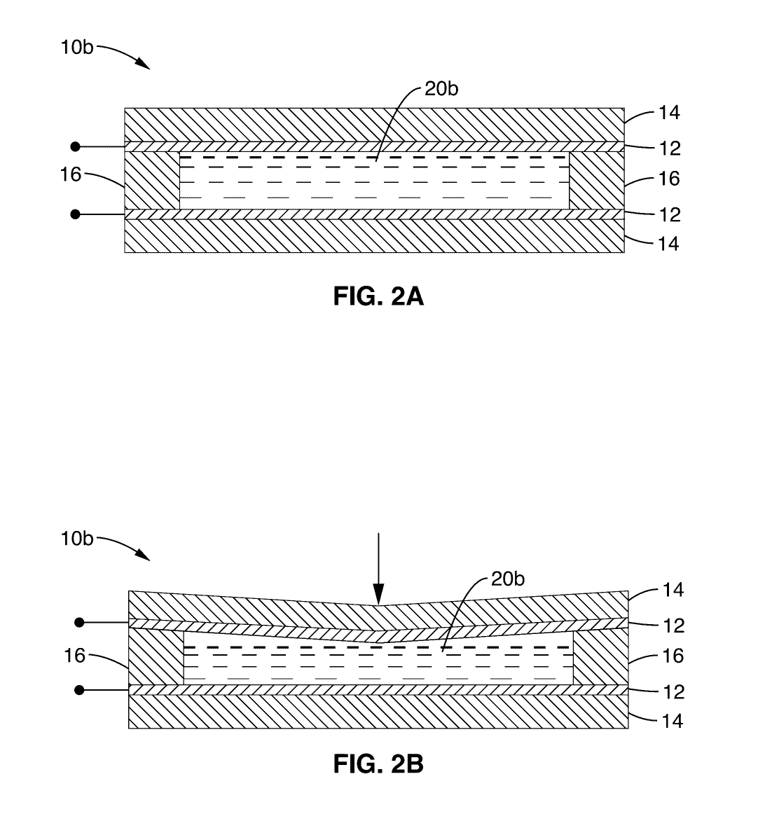

FIG. 2A and FIG. 2B are schematic diagrams of a liquid-based impedance pressure sensor 10b comprising a wholly-filled liquid chamber in initial and compressed configurations, respectively. Pressure sensor 10b comprises flexible top and bottom electrode layers 12 separated by a liquid layer 20b and mechanically supporting material 16. Electrodes 12 define opposing ends of a chamber 18, which is filled with liquid 20b such as an electrolyte solution. Additional building material may be disposed in substrate/membrane layers 14. As shown in FIG. 2A and FIG. 2B, the liquid 20b occupies the entire volume of chamber 18, as opposed to embodiment 10a of FIG. 1A and FIG. 1B.

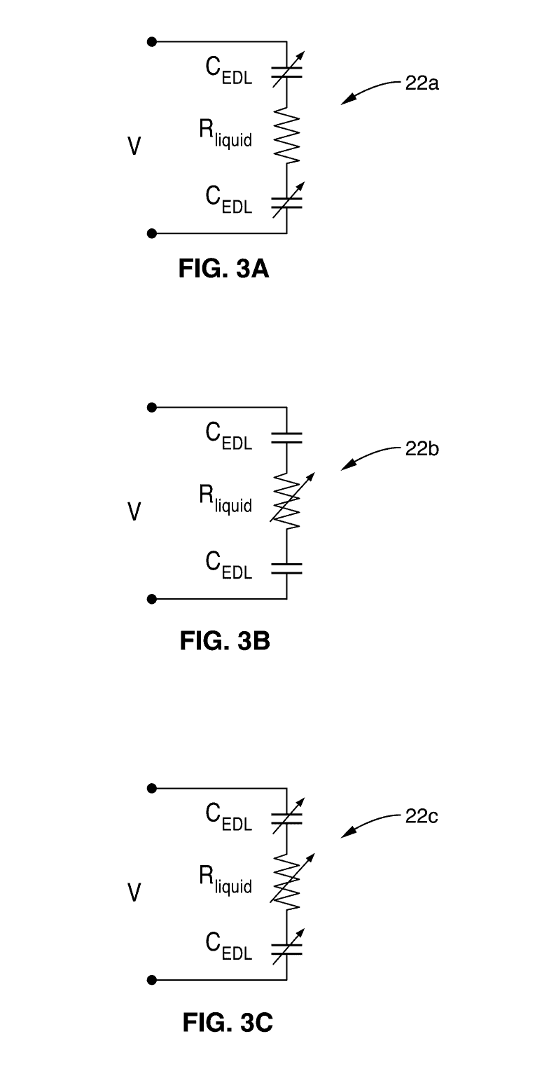

FIG. 3A shows a schematic circuit diagram of a liquid-based impedance pressure sensor in a capacitive mode 22a in accordance with the present description. Given a relatively constant charge density and voltage applied according to the capacitive mode 22a, the variation in the contact area will lead to a proportional change in the interfacial capacitance.

FIG. 3B is a schematic circuit diagram of a liquid-based impedance pressure sensor in a resistive mode 22b in accordance with the present description. Upon the contact with an external load, the force-sensing membranes 14 experience shape deformation, which results in the displacement of the fluidic sensing layer 20, as well as the change of the corresponding resistive values between the overlapped electrodes 12.

FIG. 3C is a schematic diagram of a liquid-based impedance pressure sensor in an impedance mode 22c in accordance with the present description. Upon the contact with an external load, the force-sensing membrane experiences shape deformation, which results in the displacement of the liquid layer. The area of the liquid/solid contact and the height of the liquid film change with the displacement of the liquid layer, as well as the change of the corresponding capacitive and resistive values (and thus, the impedance) between the overlapped electrodes.

In the embodiments shown in FIG. 1A through FIG. 2B, the EDL forms immediately at the liquid/solid interface, upon the liquid 20-electrode 12 contact, with mobile electrons migrating from the conductive membrane surface 12 and a counter-ion layer accumulated from the electrolyte solution (liquid) 20. The large interfacial capacitances form at the EDL layer with nanoscale separation charge.

As shown in FIG. 1A through FIG. 2B, an embodiment of the sensors 10a/10b comprises at least one EDL formed by the liquid 20 (e.g., a liquid column, typically in the range of 10 pL to 50 μL high) in contact with a conductive material layer 12 that serves as a sensing electrode (typically in the range of 1 nm to 10 μm thick). The sensors 10a/10b comprise at least one deformable chamber/cavity 18 that typically has a diameter in the range of 10 μm to 2 cm. The deformable chamber/cavity 18 may be defined by at least one deformable membrane 14 (typically in the range of 1 μm to 500 μm) supported by a supporting layer 16 (typically in the range of 1 μm to 1000 μm). In a preferred embodiment, the sensors can response to pressure/force stimuli (typically in the range of 0 MPa to 10 MPa). The change of the pressure/force will result in the contact area change of the EDL interface, and the change of the capacitance measured through the sensing electrode.

Preferably, the capacitive sensing capabilities are based on the area change of the EDL capacitor, but the sensors may also be configured to operate via other ways: e.g., by changing the distance between electrodes, the overlapping area of the electrodes or the electrical field between the electrodes.

The following description details an exemplary method for fabricating a liquid-based impedance pressure sensor in accordance with the present description. It is appreciated that the process steps and materials used are specified for exemplary purposes only, and other processes and materials may be used as available in the art.

The fabrication process starts with micropatterning of conductive indium-tin-oxide (ITO, e.g., approximately 100 nm thick) electrodes (e.g., electrodes 12) on to flexible polyethylene terephthalate (PET) films (e.g., various membrane/substrate 14 thickness from 75 μm to 175 μm) using standard photolithography, followed by wet etching.

In a subsequent step, a dry-film photoresist (50 μm, PerMX3050, DuPont) is thermally laminated onto the ITO-patterned PET substrate. Following a soft bake at 115° C. for 5 minutes, it is then exposed to selective UV lights in a mask aligner (365 nm, 220 mJ cm⁻², ABM, Inc.). In the subsequent step, the dry-film is post-baked at 95° C. for 2 minutes and developed in an ultrasonic bath with propylene glycol monomethyl ether acetate (PGMEA>99.5%) for 30 seconds, leaving the micropillar patterns on the substrate. To accurately position the microdroplets 20, a surface wettability patterning technique has been utilized. The ITO-patterned substrate is first activated with hydroxyl groups for 30 seconds in an oxygen plasma at 90 W (FEMTO). Then, a hydrophobic oligomer layer of polydimethylsiloxane (PDMS) is contact-printed onto both electrode surfaces for 2 hours, using a PDMS stamp made from a mixture of a base and a curing agent at 15:1 weight ratio. As a result, a nanometer-thick layer of PDMS oligomers is selectively deposited, forming high-contrast surface-energy patterns on the electrode 12 surfaces. Subsequently, using a microfluidic impact printing technique, nanoliter droplets (approximately 3 nL) of the ionic liquid 20 is sequentially deposited onto an array of hydrophilic microdots formed by the wettability patterning. Prior to the final assembly, two electrode films are aligned face-to-face with the conductive patterns positioned orthogonally to each other, forming a grid of capacitance at the crossover points where the ionic droplet array sits in. The top and bottom layers are then bonded together after the oxygen-plasma activation of hydroxyl groups of the PDMS oligomer layers (30 second exposure at 90 W).

In preferred embodiments, the sensing liquid 20 preferably comprises high conductivity, low evaporation under normal condition, low viscosity, and high surface tension liquids. Combined with these characteristics, an ionic liquid is an ideal choice for the sensing liquid 20. The sensing liquid may also be other materials, including: an inorganic material, water-based salt solution (e.g., KCl-water), liquid metal (EGaln, Hg), polar molecular liquid (e.g., ethylene glycol) or organic solvent based salt (e.g., KCl-Methanol).

The sensing liquid 20 may be an ionic polymer, composite, or nanomaterials or other soft-matter materials. The sensing liquid 20 may also be in the gel state, such as hydrogel polymer.

The conductive material for electrode 12 may comprise one or more of the following materials: a conductive material: metal (gold, liquid metal), metal alloy (ITO), conductive polymer (PEDOT:PSS), carbon-based material (e.g., CNT, graphene, carbon black), or conductive nano-structured conductive material (e.g., Ag NW, NT).

The conductive material for electrode 12 may comprise a material coated with a conductive material: e.g., polymer, silicon, or glass.

The building materials (e.g., substrate/membrane layers 14, supporting layer 16) for the sensor may comprise silicon, polymer, metal, glass, semiconductor, etc.

Exemplary bonding materials may comprise a polymer, such as Avatrel, PPA BCB (Benzocyclobutene), silicone (PDMS), Polyimide, SU-8, or PMMA. The bonding methods for the device package can be: adhesive bonding, anodic bonding, plasma activated bonding, direct bonding (technique in silicon wafer bonding), ultrahigh vacuum bonding, etc.

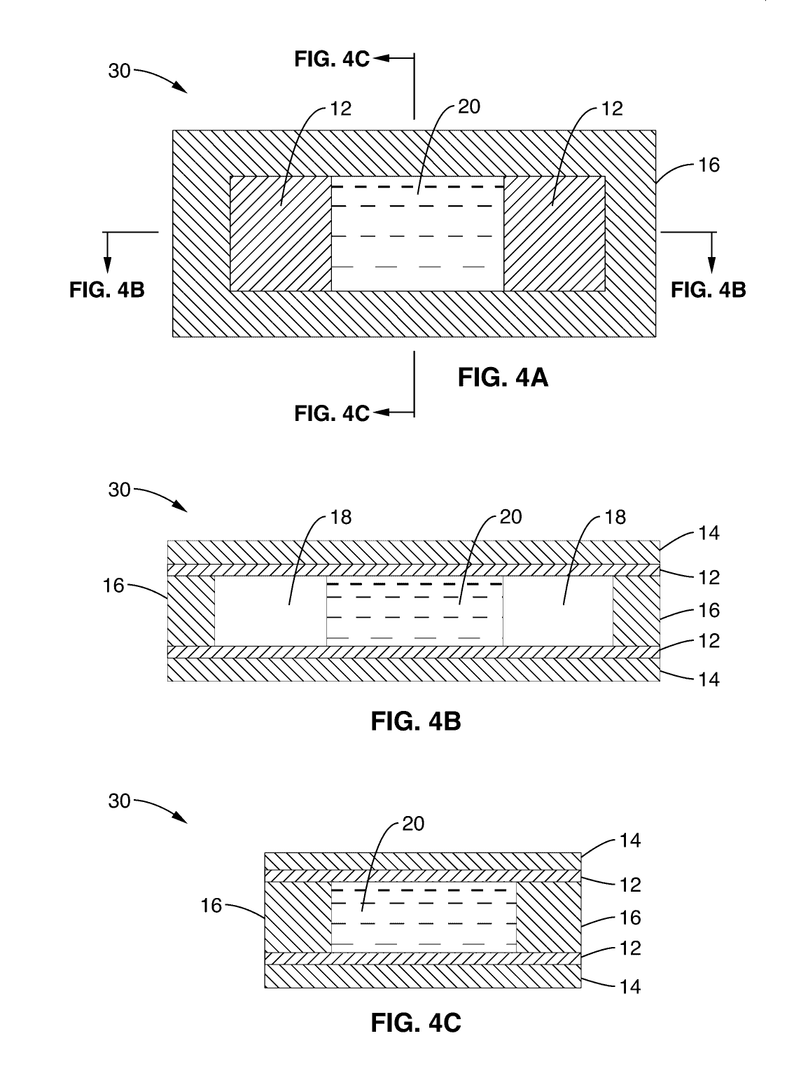

FIG. 4A through FIG. 22B show additional configurations of liquid-based impedance pressure sensors utilizing differing functionality and geometry. It is appreciated that any of the principles disclosed above for FIG. 1A through FIG. 3C may interchangeably be applied to the devices shown in FIG. 4A through FIG. 22B.

FIG. 4A through FIG. 4C illustrate a liquid-based impedance pressure sensor 30 having a cavity 18 that is formed by three layers of building material: top and bottom substrate layers 14 with one supporting layer 16 in between. FIG. 4A shows a plan section view with the top layers removed, and FIG. 4B and FIG. 4C are orthogonal sections of the view of FIG. 4A. In the configuration of pressure sensor 30, the liquid 20 fills half of the cavity 18 (see FIG. 4B), and forms a liquid column 20 that contacts with electrodes 12 of top and bottom substrates 14, forming two solid-liquid interfaces; the solid-liquid interface of the liquid will have 1D movement along the cavity 18. The sensing electrodes can be on one of the interface.

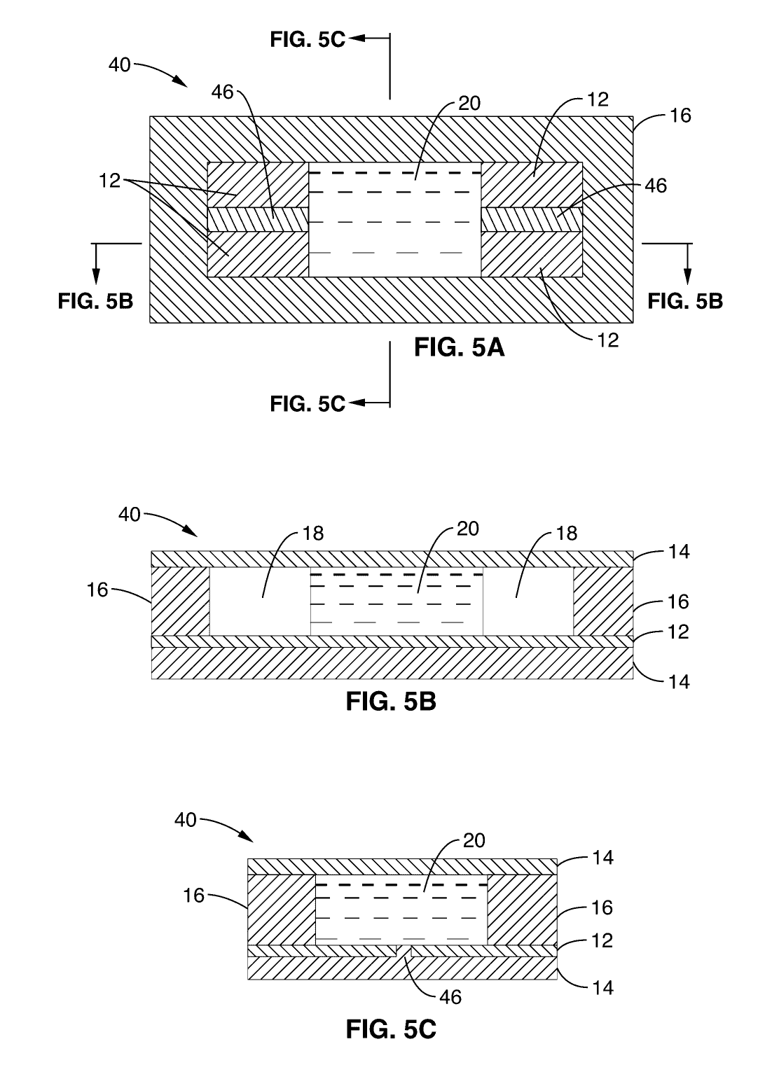

FIG. 5A through FIG. 5C illustrate a liquid-based impedance pressure sensor 40 having a cavity 18 that is formed by three layers of building material: top substrate layer 14 and bottom layer 14 separated by supporting layer 16, and only one electrode layer 12. FIG. 5A shows a plan section view with the top layers removed, and FIG. 5B and FIG. 5C are orthogonal sections of the view of FIG. 5A. Electrode layer 12 is split into two co-planar electrodes via a longitudinal protrusion 46 running along the length of bottom substrate layer 14.

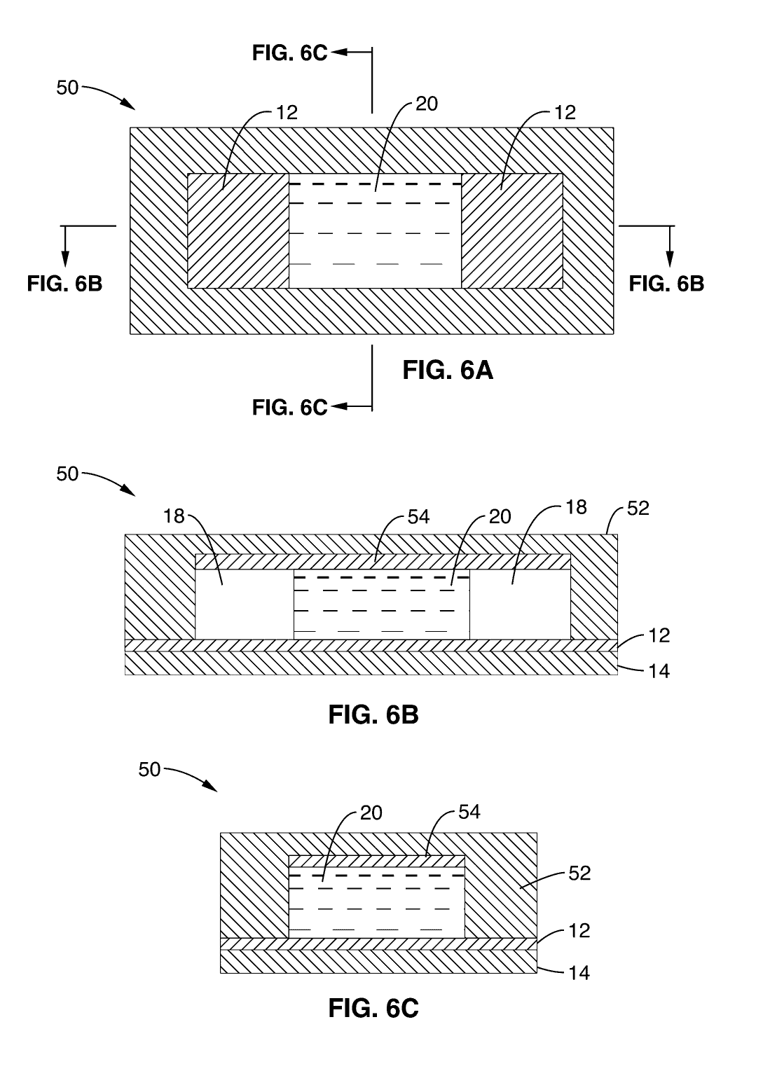

FIG. 6A through FIG. 6C illustrate a liquid-based impedance pressure sensor 50 having a cavity 18 that is formed by two layers of building material: bottom substrate layer 14 and top layer 52 that is u-shaped to form cavity 18. FIG. 6A shows a plan section view with the top substrate layer removed, and FIG. 6B and FIG. 6C are orthogonal sections of the view of FIG. 6A. Note that this orientation may be switched, e.g., with the cavity formed in bottom layer 52. The liquid 20 is hosted in the cavity 18 in contact with upper electrode 54 disposed in the cavity 18 of top substrate 52, and lower electrode 12 in contact with bottom substrate 14 to form two solid-liquid interfaces. The solid-liquid interface of the liquid column 20 is free to have 1D movement along the cavity. The sensing electrodes can be on one of the interfaces or on both interfaces.

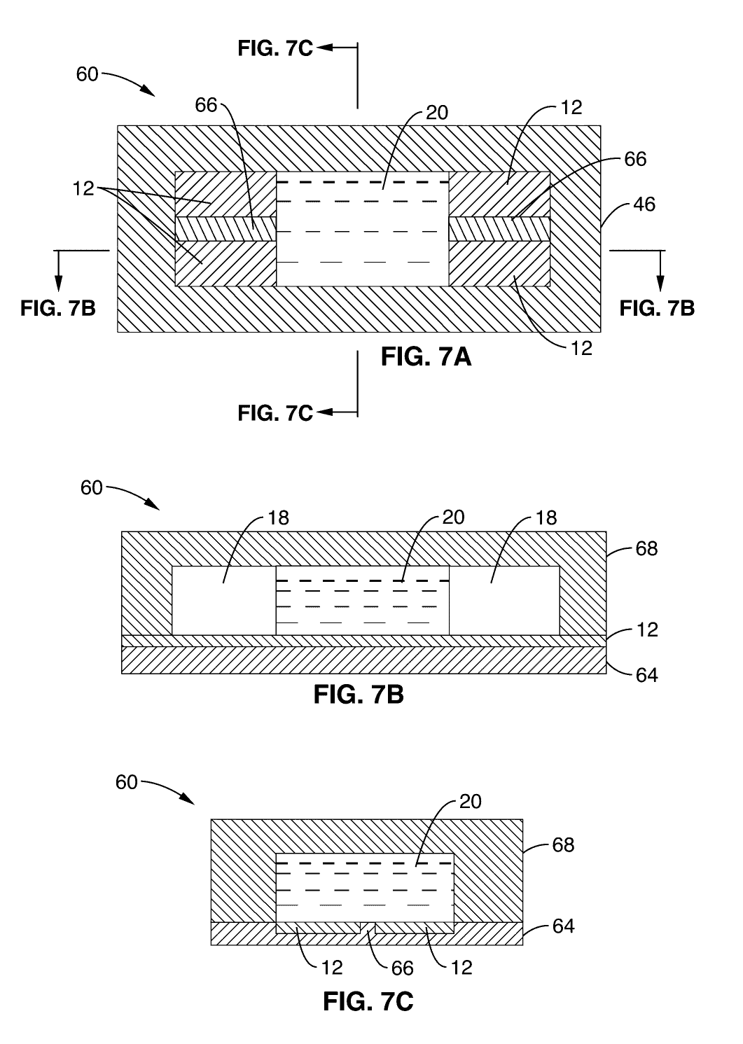

FIG. 7A through FIG. 7C illustrate a liquid-based impedance pressure sensor 60 having only one electrode layer 12 and a cavity 18 that is formed by two layers of building material: bottom substrate layer 64 and top substrate layer 68 that is u-shaped to form cavity 18. FIG. 7A shows a plan section view with the top electrode and upper section of top substrate layers removed, and FIG. 7B and FIG. 7C are orthogonal sections of the view of FIG. 7A. Note that this orientation may be switched, e.g., cavity formed in bottom layer 64. The liquid 20 is hosted in the cavity 18 of top substrate layer 68 Electrode layer 12 is split into two co-planar electrodes via a longitudinal protrusion 66 running along the length of bottom substrate layer 64.

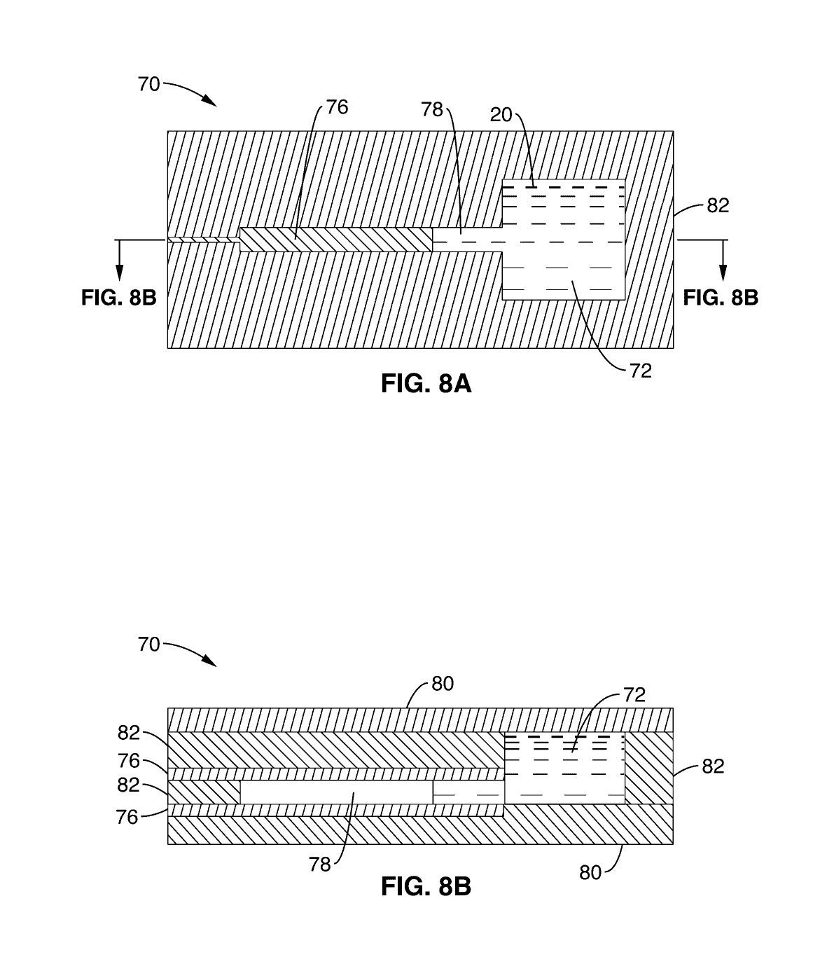

FIG. 8A through FIG. 8B illustrate a liquid-based impedance pressure sensor 70 comprising a chamber 72 and a channel 78 that is formed via top and bottom substrate layers 80, and supporting layers 82. FIG. 8A shows a plan section view with the top electrode and upper substrate layers removed, and FIG. 8B is an orthogonal section of the view of FIG. 8A. Electrodes 76 are disposed on opposing sides of the channel 78. Without application of an external load, the liquid 20 is stored up (substantially or entirely) in the chamber 72. When the external load is applied to the membranes 80 of the chamber 72, the liquid 20 expands into the channel 78, and the contact area of the electrolyte-electrode interface increases.

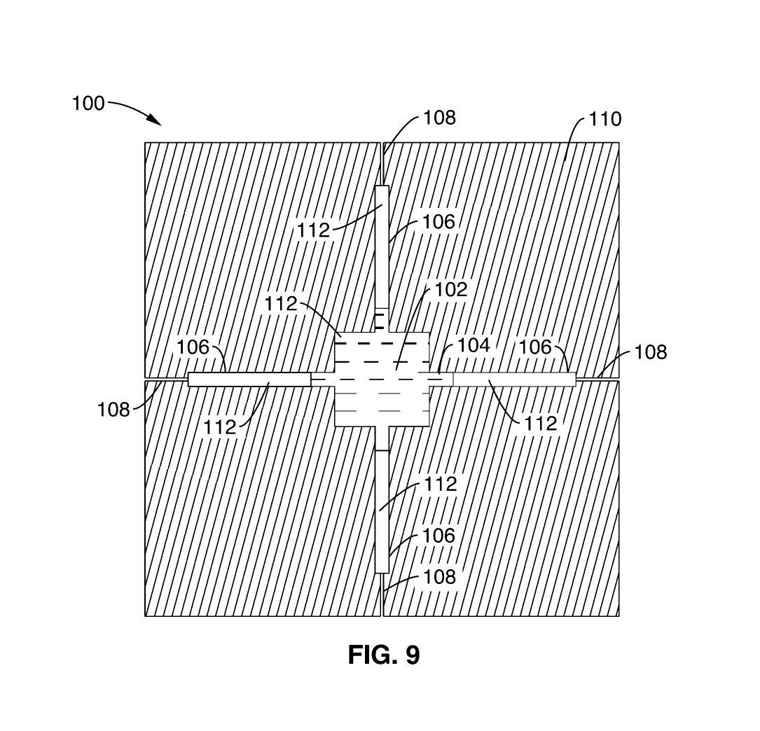

FIG. 9 illustrates a top section view of a liquid-based impedance pressure sensor 100 comprising a chamber 102 and a plurality of channels 106 formed via one or more upper substrate layers (not shown) and supporting layer 110, similar to that shown in FIG. 8A through FIG. 8B. Electrodes 112 are disposed on opposing sides of the channel 106 (a single layer of coplanar electrodes may also be used, as shown in FIG. 15A and FIG. 15B). Without application of an external load, the liquid 104 is stored up (substantially or entirely) in the chamber 102. When the external load is applied to the membranes of the chamber 102, the liquid 104 expands into the channel 106, and the contact area of the electrolyte-electrode interface increases. Ports 108 allow air into the channels 106 to allow for the liquid 104 to be freely displaced. The liquid 104 position can be in the center as a column shown in several of the embodiments above, the entire chamber (FIG. 2A and FIG. 2B), or at one side of the chamber.

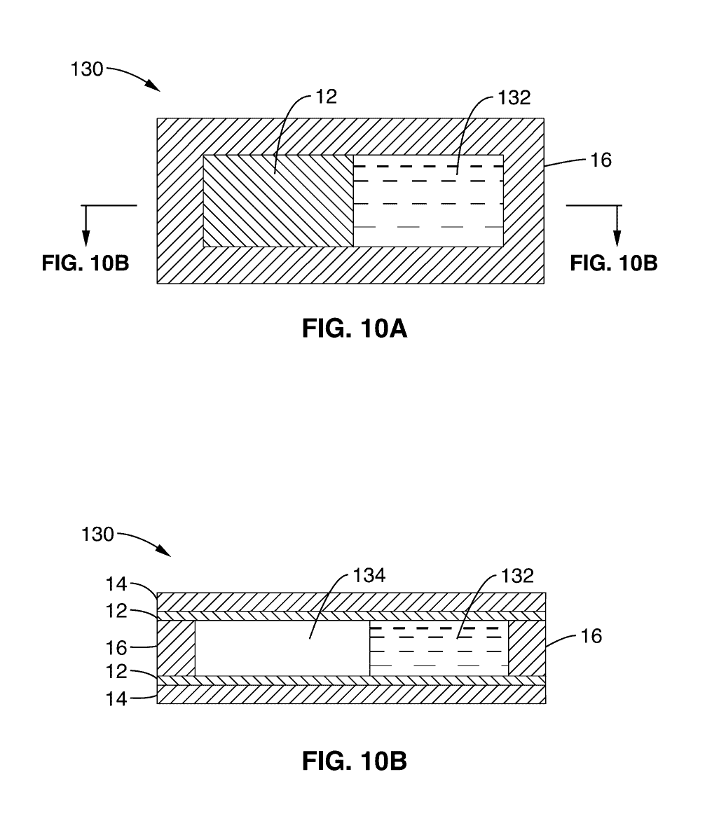

As shown in the liquid-based impedance pressure sensor 130 of FIGS. 10A and 10B, liquid 132 is disposed on the right side of chamber 134, such that liquid primarily contacts one end of the electrodes 12. FIG. 10A shows a plan section view with the top electrode and upper substrate layer removed, and FIG. 10B is an orthogonal section of the view of FIG. 10A.

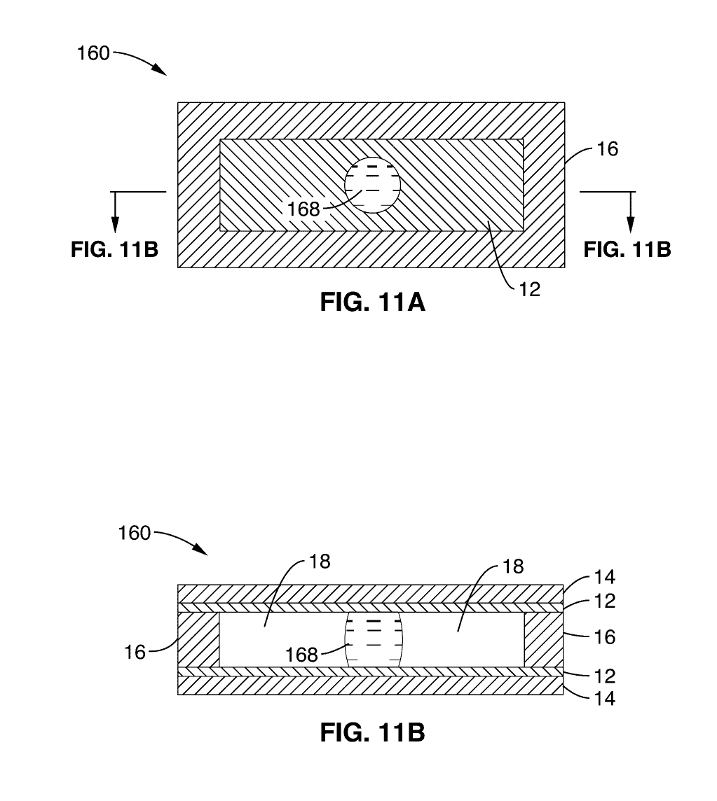

Furthermore, as shown in the liquid-based impedance pressure sensor 160 of FIG. 11A and FIG. 11B, the liquid may be formed as a droplet 168 disposed within a location of chamber 18. FIG. 11A shows a plan section view with the top electrode and upper substrate layer removed, and FIG. 11B is an orthogonal section of the view of FIG. 11A.

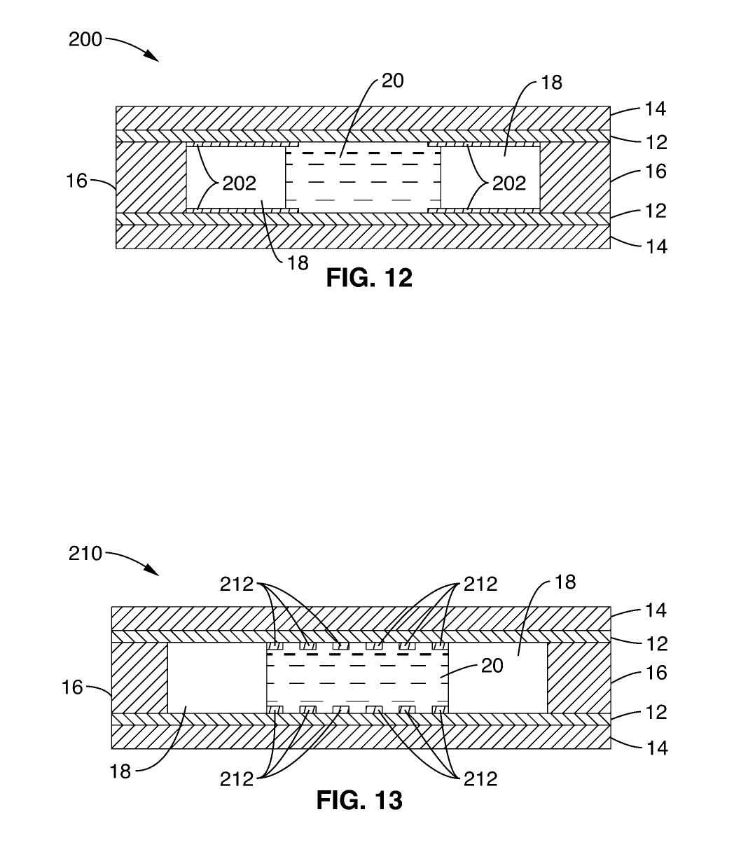

Referring to the liquid-based impedance pressure sensor 200 of FIG. 12, an anchor structure may be used for locking the location of the liquid 20 within chamber 18. This can be achieved by patterning the substrate 14/electrode 12 of the sensor 200 with a hydrophobic area or region 202 to repel the liquid 20 into a specified location (which is correspondingly hydrophillic and can be circular, rectangular, centered, left/right justified, etc.).

Referring to the liquid-based impedance pressure sensor 210 of FIG. 13, an alternative anchor structure may be used for locking the location of the liquid 20 within chamber 18. This can be achieved by patterning the substrate 14/electrode 12 of the sensor 210 with a micropillar structure 212 at the center or other location of the chamber 18. The micropillar structure 212 acts to retain the liquid 20 at a desired location, and thus functions as a hydrophillic region. The micro-pillar structures 212 can be grown on at least one of the top and bottom surface to avoid shorting of sensor. The number of the micro-pillars 212 can be single or multiple.

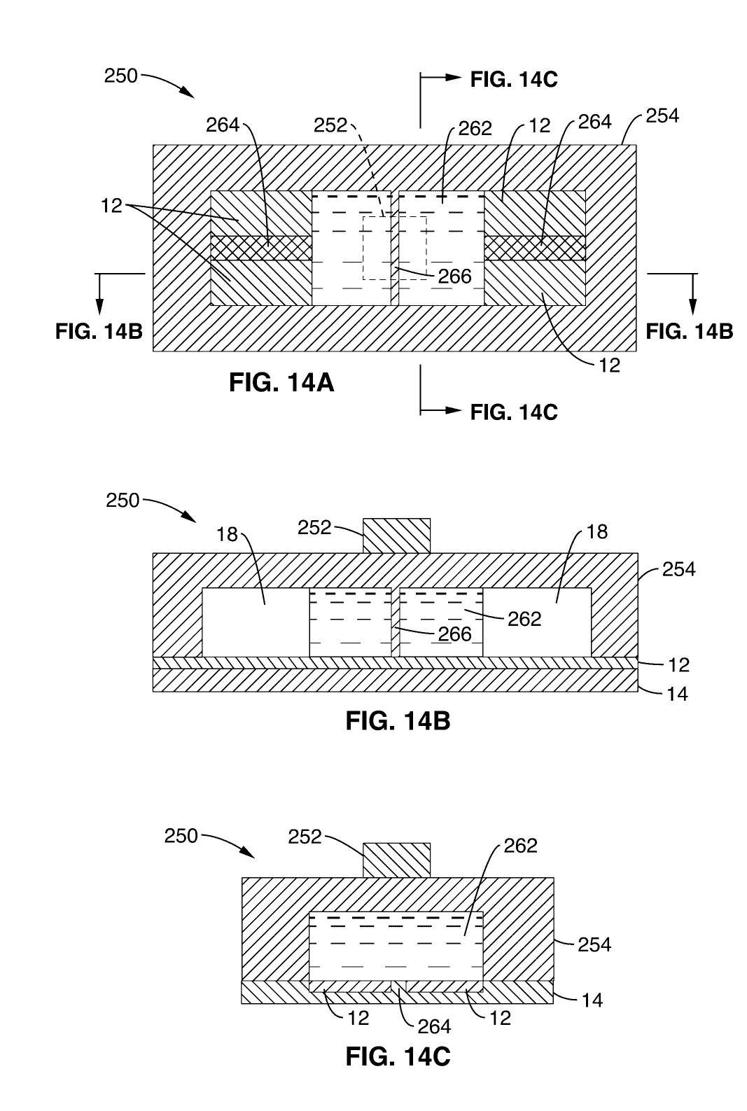

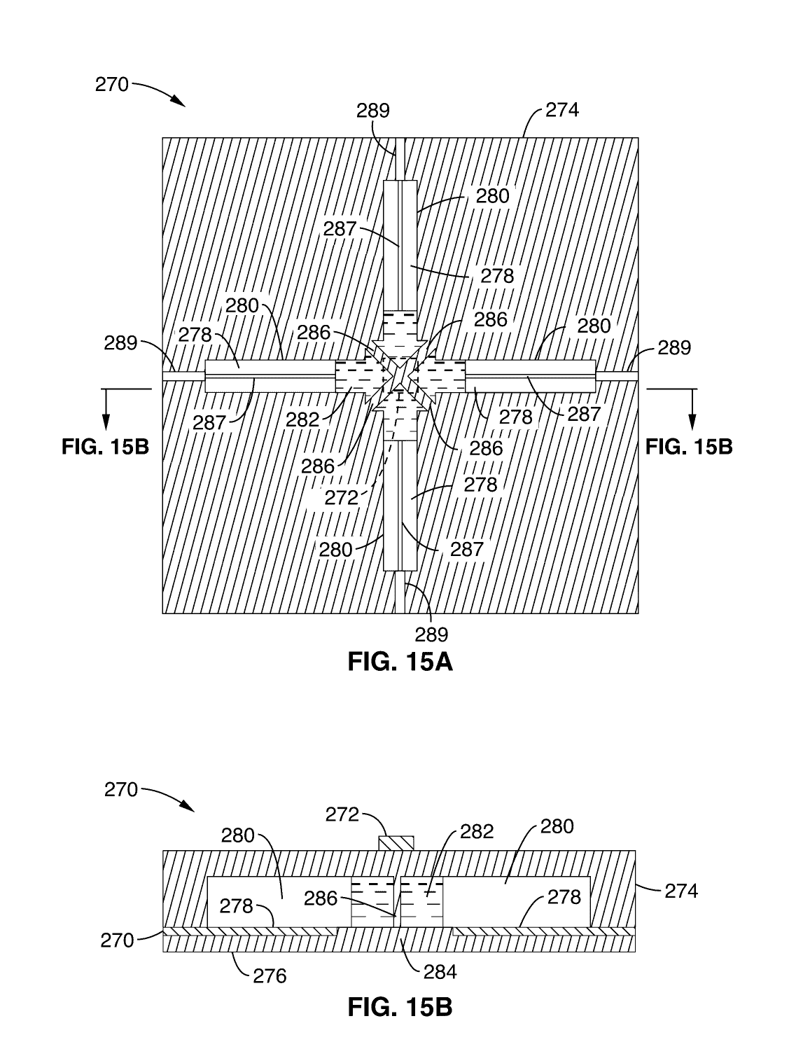

Referring to the liquid-based impedance pressure sensor 250 of FIG. 14A through FIG. 14C, a bump structure 252 can be applied to the liquid-based pressure sensor 250 for detection of both normal and shear force measurements. FIG. 14A shows a plan section view with the top electrode and upper section of top substrate layer 254 removed, and FIG. 14B and FIG. 14C are orthogonal sections of the view of FIG. 14A. One or more dividing support layers 266 may be included to provide separation between sides of chamber 18, and therefore liquid 262 within said chamber 18. Electrodes 12 may be disposed in a coplanar configuration via an additional linear protrusion (e.g. similar to protrusion 46 of FIG. 5C), or in an opposing configuration similar to electrodes 76 shown in FIG. 8B), thus allowing for independent sensing between left and right sides of chamber 18 (e.g. with four electrodes, first and second electrodes independently sensing with respect to third and fourth electrodes). A microbump 252 can be applied to the upper substrate layer 254 (which forms a u-shaped cavity 18 for holding liquid 262), to detect the normal force and one-dimensional shear force via expansion of liquid 262 with electrodes 12 formed from lower substrate layer 14 and protrusion 264.

Referring to the liquid-based impedance pressure sensor 270 of FIG. 15A through FIG. 15B, a bump structure 272 can be applied to the liquid-based pressure sensor 270 for detection of both normal force and two-dimensional shear force. FIG. 15A shows a plan section view with the top electrode and portion of upper substrate layers removed, and FIG. 15B is an orthogonal section of the view of FIG. 15A. Pressure sensor 270 comprises a four-channel chamber 280 formed via upper substrate layer 270 and lower substrate layer 276. Electrodes 278 are disposed in the same layer at opposing sides of protrusion 284 to line the bottom of each channel in chamber 280. Electrodes 278 may be disposed in a coplanar configuration via a linear protrusion 287 running along each channel of chamber 280 (or, alternatively, in an opposing configuration similar to electrodes 76 shown in FIG. 8B). Thus, each channel in chamber 280 will have a pair of independent electrodes that are capable of individually sensing fluid 282 level in each channel of chamber 280 (e.g. first and second electrodes in the left part of channel of chamber 280 independently record data from third and fourth electrodes in right channel of chamber 280).

Supporting material 286 may be added to the chambers for additional support, and for separating the liquid 282 into each of the four channels of chamber 280. Thus, the liquid 282 acts independently for each channel of chamber 280. Each channel of chamber 280 may also include a port 289 to allow for the liquid 282 to be freely displaced.

When a normal force is applied to the bump 272, the liquid 282 will go into the four detecting channels of chamber 280 equally, resulting in a same increase of the capacitance values; a shear force (e.g. when the direction of the force is applied laterally from left side to the right side in FIG. 15B) will cause the torque of the bump 272, resulting the increase of the capacitance value in the right channel and the decrease of the capacitance value in the left channel. The other two values (for top and bottom channels of chamber 280) will remain the same. The amplitudes and the directions of the external forces can be back-calculated from the changes of the capacitance values in the four detecting channels 288.

The wettability of the substrate can be modified, such as by oligomer coating, SAM coating, silane modification, PEG coating, oxygen plasma activation, piranha modification, super-hydrophobic spray coating, corona, sputtering. The center spot can be modified to be hydrophilic to anchor the droplet.

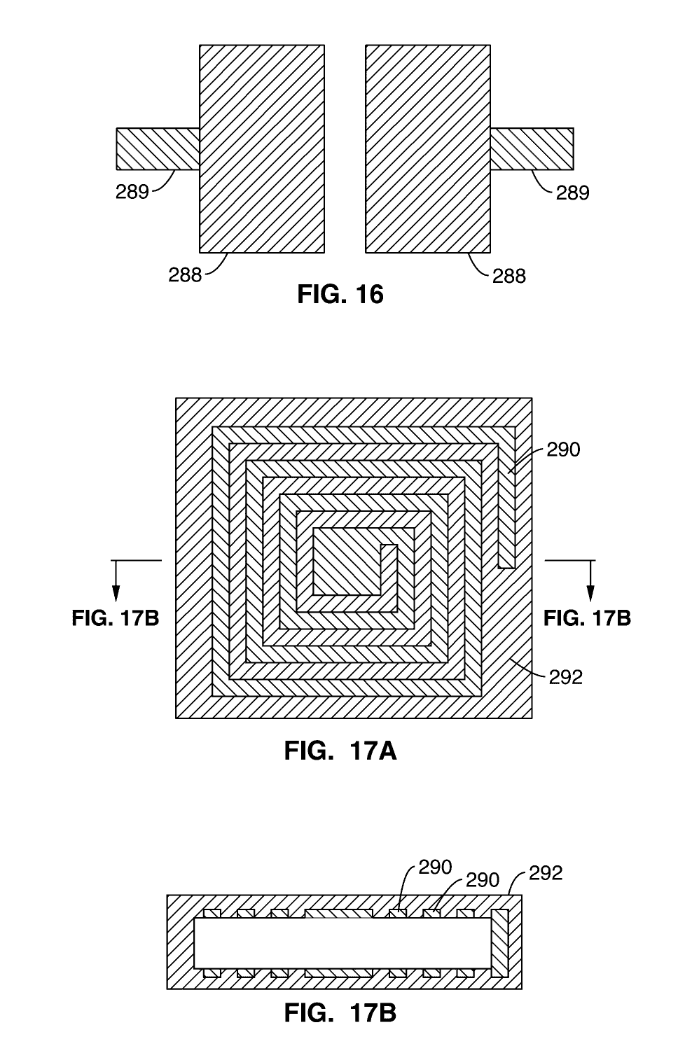

The sensing electrodes (which may be disposed on one of the interfaces or on both interfaces) can be in various geometries, including co—the planar electrodes 288/289 shown in FIG. 16.

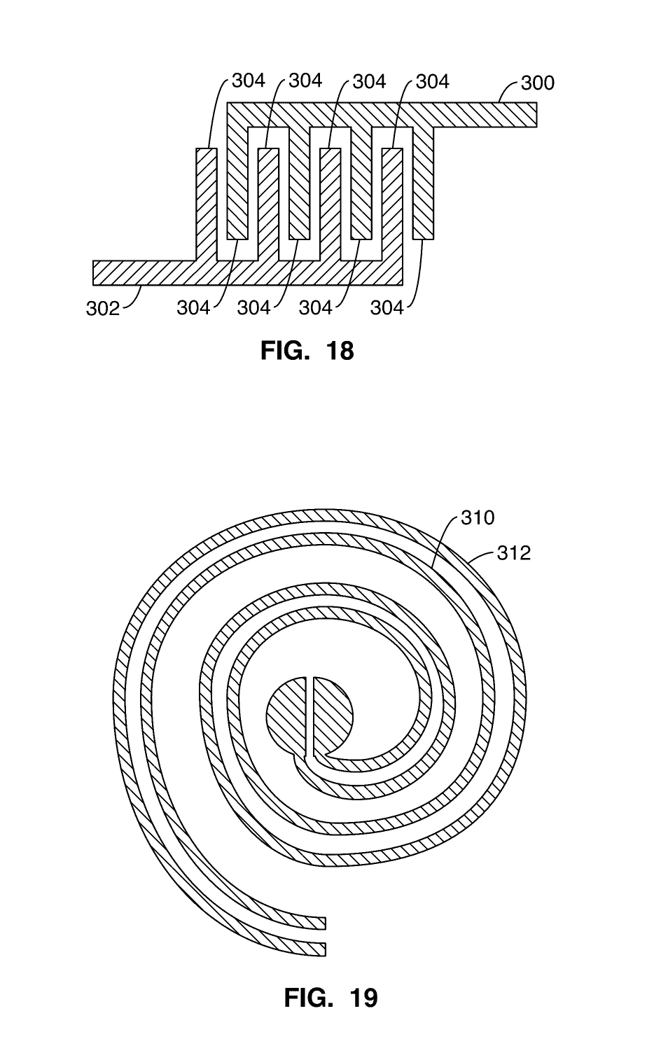

Other electrode configurations included opposing patterns 290 (planar coils comprising of a few turns of microelectrodes) on substrate 292 shown in FIG. 17A and FIG. 17B, and interdigitated electrodes 300/302 with interlaced tines 304 (FIG. 18) and spiral electrodes 310, 312 (FIG. 19) et al.

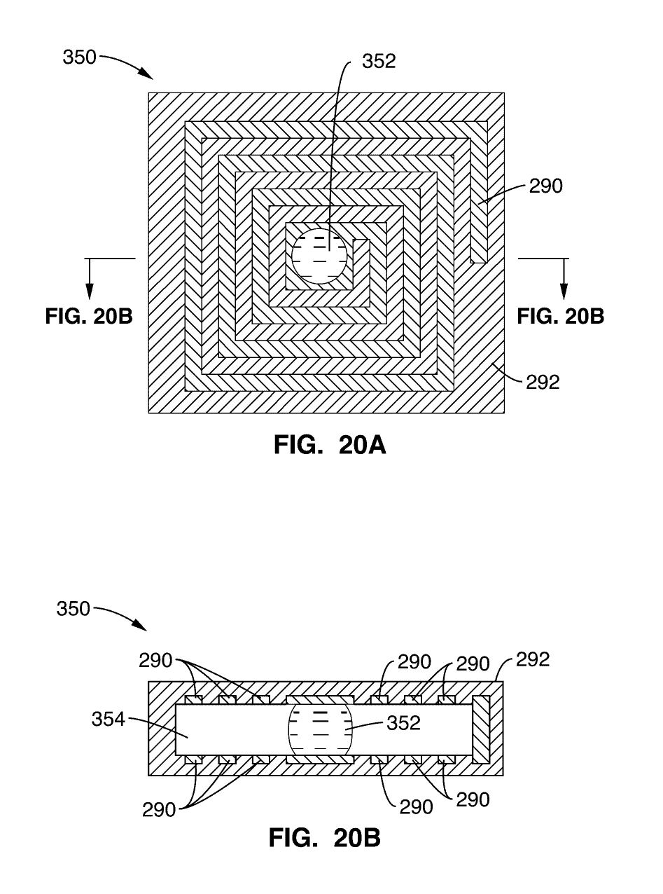

An exemplary liquid-based LC wireless pressure sensor 350 is shown in FIG. 20A and FIG. 20B. The capacitive element comprises a sealed microcavity 354 with a sensing droplet 352 sandwiched by two electrode plates 290 having the pattern of FIG. 17A and FIG. 17B. Small deflections of the electrode plates 290 due to ambient stress change the resonant frequency of the LC circuit, which can be detected by an external coupling antenna to the coil inductor.

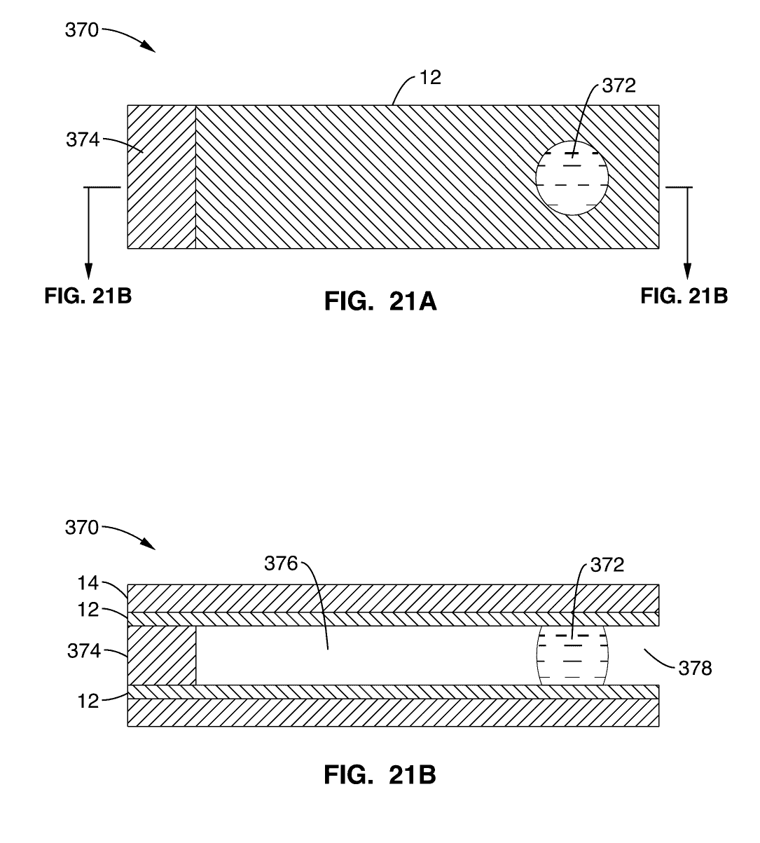

FIG. 21A and FIG. 21B illustrate a liquid-based impedance pressure sensor 370 having a cavity 376 that is formed by three layers of building material: top and bottom substrate layers 14 with one supporting layer 374 in between the substrate layers only one side of the chamber 376. FIG. 21A shows a plan section view with the top layers removed, and FIG. 21B shows an orthogonal section of the view of FIG. 21B. The sensor 370 thus has a cantilevered open end 378. A droplet of sensing liquid 372 is positioned at the open end 378 of the chamber 376, which is supported by the droplet 372. Sensing liquid 372 contacts top and bottom electrodes 12 and forming two interfacial capacitance locations. The liquid 272 will expand with the deformation of the cantilever chamber 376 under pressure.

In another embodiment (not shown), the supporting layer is only placed at the two opposing ends of the chamber 18, leaving two ends open. In such configuration liquid 272 is positioned in the center of the chamber.

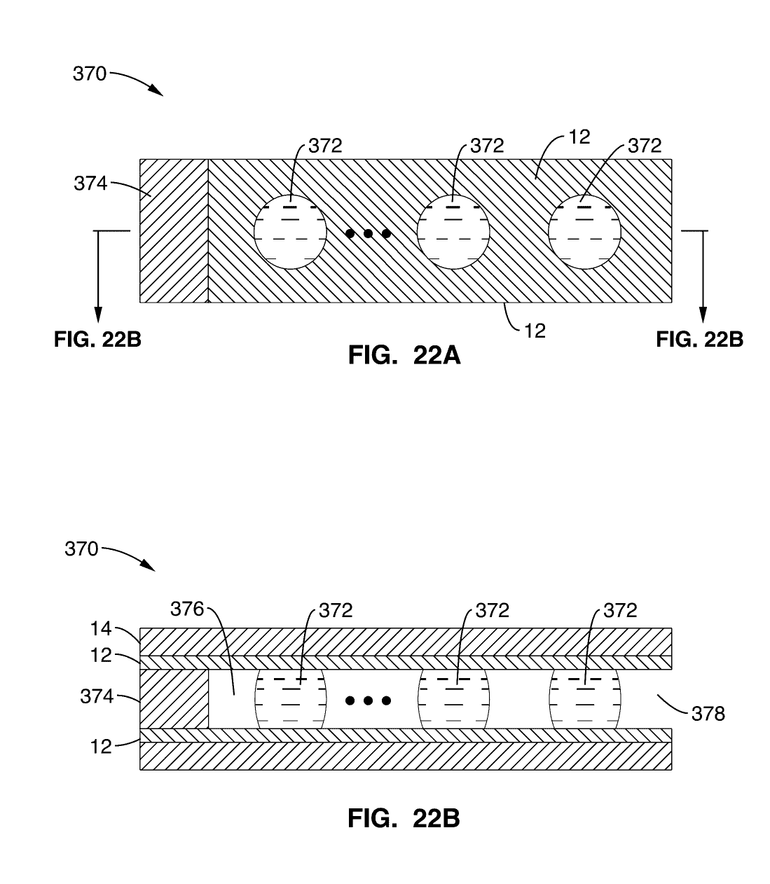

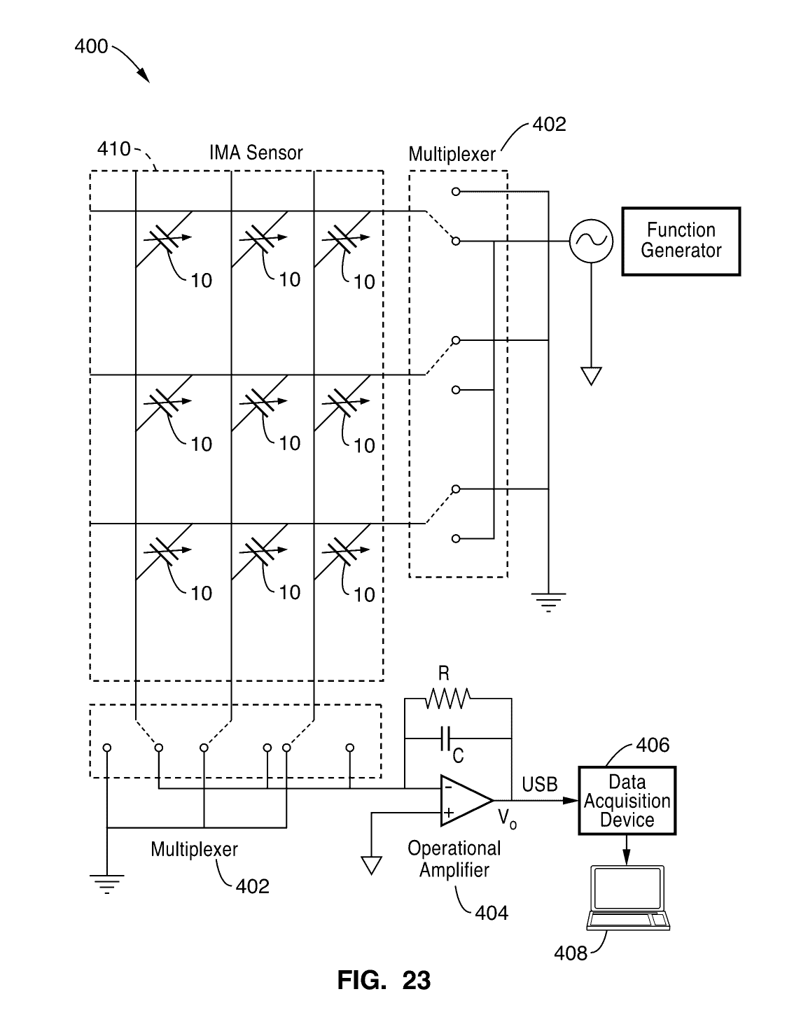

FIG. 22A and FIG. 22B illustrate a liquid-based impedance pressure sensor 390 having a cavity 376 that is formed by three layers of building material: top and bottom substrate layers 14 with one supporting layer 374 in between the substrate layers only one side of the chamber 376. FIG. 22A shows a plan section view with the top layers removed, and FIG. 22B shows an orthogonal section of the view of FIG. 22B. The sensor 390 has a cantilevered open end 378 with a plurality of droplets of sensing liquid 372 positioned at spaced-apart locations within chamber 376. Sensing liquid 372 contacts top and bottom electrodes 12 and forming two interfacial capacitance locations. The liquid droplets 272 may all variably expand with the deformation of the cantilever chamber 376 under pressure.

FIG. 23 is a schematic diagram illustrating exemplary measurement system 400 configured to assess the electrical readouts from the IMA devices. The system 400 comprises a pixel selection unit 412 a signal amplification unit 404, and a data acquisition unit 406. All of the individual pixels (e.g., sensors 10 of array 410) can be addressed by two orthogonally controlled multiplexers 402 that are regulated by a microcontroller/function generator 412. Once a pixel 10 is selected, the output signal (V₀) goes through operational amplifier 406 and is measured by a 16-bit data acquisition module 406. Data acquisition algorithms may be programmed and the recorded data processed via a computer processor unit 408.

### Example

Experimental investigations of the sensing device of the present disclosure were conducted on individual sensing units of the iontronic microdroplet array. A measurement stage comprising of a force gauge with 1 mN resolution mounted on a computer-controlled step motor with a spatial resolution of 400 nm was used for simultaneously controlling and monitoring mechanical loads and displacements. Pressure values were calculated based on the ratio of the applied force to the surface area of the membrane in each sensing unit. The corresponding capacitive changes were directly recorded through an LCR meter. Each sensitivity measurement was conducted twice on two identical sensing devices. In the characterization of the responsive time, an electromagnetically driven pin actuator, powered by a pulsed voltage signal from 1 Hz to 100 Hz, was used to apply the periodic contact pressure to the sensor surface. The output signals of the IMA device are measured by use of the readout circuitry of FIG. 23.

Iontronic capacitive sensing is generally established upon forming an ionic-electronic interface at the droplet-electrode contact. The droplet sensing fluid preferably meets several design criteria, including high conductivity (providing ultrahigh EDL capacitance and low electrical loss), low viscosity (ensuring short response time) and electrochemical stability (no electrochemical reaction under the operating voltage), environmental stability (maintaining the physical properties over the operating period). Three types of ionic fluids have been considered, such as aqueous electrolytes (e.g., NaCl electrolyte solution), organic solvent solutions (e.g., KClO₄/PEO), and ionic liquids, which are commonly investigated in electrochemical processes. Aqueous and solvent-based electrolytes are typically highly evaporative under room conditions, thus making it extremely challenging to maintain the constant electrical performance, as both the volume and the physical properties change over time. Ionic liquids (ILs), comprising of an organic anion or cation, exhibit high electrical conductivity, low volatility, and tunable viscosity. In addition to its wide electrochemical window, ILs are the ideal candidates for the microdroplet sensors.

Several types of imidazolium-based ILs were contemplated in the device of the present description, including 1-butyl-3-methylimidazolium hexafluorophosphate, 1-butyl-3-methylimidazolium tetrafluoroborate, 1-ethyl-3-methylimidazolium tetrafluoroborate, and 1-ethyl-3-methylimidazolium tricyanomethanide, which are a group of ILs that show excellent ionic conductivity and good chemical stability. The above listed ILs have wide chemical window, ranging from 2.6V to 5.7V, and possess negligible vapor pressures (more than thousand times lower than water). In addition, the low melting points of the ILs (typically less than 0° C.) ensure the liquid state under room temperature conditions. Interestingly, the electrical conductivities of the ILs are inversely related to the dynamic viscosities, which allows for their use in ultrasensitive and highly responsive tactile sensors. Accordingly, the ionic liquid of 1-ethyl-3-methylimidazolium tricyanomethanide, with the highest conductivity (18 mS cm⁻¹) and lowest viscosity (18 Pa·s) among the iontronic fluid of the ILs listed above, was selected as the working fluid in the iontronic droplet sensors.

The EDL structure presents a remarkable unit-area capacitance at the nanoscopic interface between the electrode and the electrolyte droplet. Unlike the solid-state capacitors, it is established by mobile electrons in a conductive surface and counter-ions immigrating in the adjacent liquid environment, and the value can be determined by the surface charge density and Debye length. In particular, the EDL capacitance is frequency-dependent with several intermolecular interaction mechanisms associated (e.g., interfacial polarization). The frequency dependence of the EDL is characterized by using a LCR meter to determine the unit-area capacitance of a symmetric ITO/IL/ITO structure in the sub-MHz spectrum.

Prior to the measurement, an IL (of 0.3 μL) droplet is sandwiched between two ITO-coated PET films, of which the conductive ITO layer is 100 nm in thickness. Under an AC excitation voltage at 0.5 V, the device is connected to the LCR meter in a bipolar configuration.

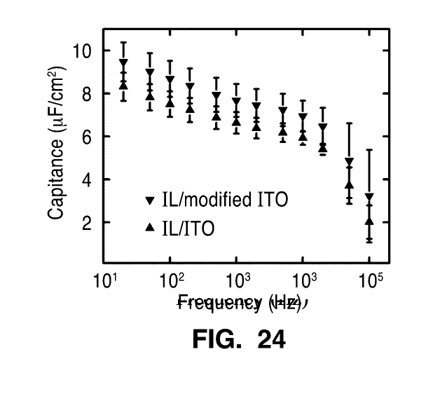

FIG. 24 plots the frequency responses of the EDL for the ionic droplets on both hydrophobic-modified and unmodified electrode surfaces, respectively. As can be seen, the EDL exhibits the maximal unit-area capacitance around 10 μF cm⁻² at DC, slowly decreases with the rising frequency until 20 kHz (6 μF cm⁻²). Then, drastically declines beyond this turning point, which mainly attributes to the low-frequency dispersion of ionic conductions. Interestingly, the modified ITO surface expresses a slightly higher unit-area capacitance than that of the unmodified one, possibly due to removal of a native oxide layer by the plasma treatment and improved interactions between ionic and electronic charges.

a. Theoretical Analysis

The device sensitivity of the iontronic microdroplet sensor can be modeled both mechanically and electrically. As aforementioned, under the external load, the suspended membranes deform elastically over the droplets, and accordingly, the droplet-electrode contact experiences circumferential expansion. The measured EDL capacitance can be directly related to the area of the droplet-electrode contact, as the invariant unit-area capacitance can be experimentally determined. On the other hand, the mechanical deformation of the membrane can be well defined in the classic mechanic theory. It is worth noting that the interfacial capacitive sensing principle offers an ultrahigh capacitive sensitivity, which is more than thousand times greater than that of the solid-state counterpart, contributed mainly from the nanoscopic charge separation in EDL, yielding ultrahigh overall device sensitivity.

The relationship between the measurable capacitive change (ΔC) and the contact pressure applied (ΔP) can be derived from the new interfacial capacitive sensing principle:

$$Δ C \approx {C}_{0} \times [ \frac{Δ P}{K} + {( \frac{Δ P}{K} )}^{2} ] \qquad Eq . 1$$

where C₀ indicates the initial capacitance, K=5ET³h/(1−ν²)a⁴ is a constant derived from the design parameters, including the width (a) and height (h) of each sensing cell, and the membrane properties, including Young's modulus E, thickness T and Poisson ratio ν. The gravitational effect has been neglected in our consideration, as the microdroplet dimensions are considerably less than that of the capillary length (of approximately 1.8 mm).

b. Experimental Characterization

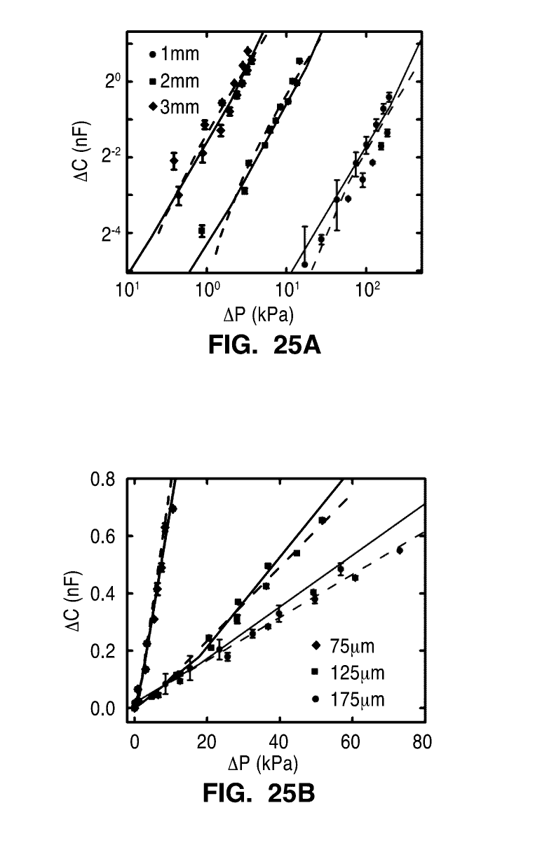

FIG. 25A and FIG. 25B show experimental measurements on the device sensitivity with various geometrical designs (i.e., spatial resolutions and membrane thicknesses), in which the measurement results (dots) with the corresponding fitting curve (dash lines) are plotted against the theoretical predications from Equation 1 (solid lines). As a result, the device sensitivity can be calculated from the slope rate of each ΔP−ΔC curve. As expected, the sensitivity exhibits a strong dependence (minor 4th power) on the spatial resolution. As shown in FIG. 25A, by varying the spatial resolution from 1 mm to 3 mm with a constant membrane thickness of 75 μm, the sensitivity can be improved from 3.9 pF kPa⁻¹ to 433.7 pF kPa⁻¹ (more than 100-fold increase), and the device achieves the highest sensitivity (of 77.7 pF kPa⁻¹) with a large initial capacitance (of 2.2 nF) in comparison with the highest reported values in the literature which has a sensitivity of 2 pF MPa⁻¹ and the initial capacitance of 14 pF at the spatial resolution of 2 mm, to the best of our knowledge. In addition, the minimal detectable pressure of 33 Pa is characterized on the sensor with the highest sensitivity.

Moreover, the membrane thickness plays another notable role in the device performance, as the sensitivity is inversely related to the 3^{rd} power of the thickness. As plotted in FIG. 25B, by adjusting the membrane thickness from 75 μm to 175 μm, with a fixed spatial resolution of 2 mm, the thinner membrane (of 75 μm-thick) shows a higher sensitivity of 77.7 pF kPa⁻¹, while the thicker devices (of 175 μm-thick) membranes exhibit a lower sensitivity of 7.8 pF kPa⁻¹. Furthermore, the targeted dynamic range can be tuned by the geometrical constrains. For instance, in the most sensitive design (3 mm in resolution and 75 μm in thickness), the maximal pressure is around 7 kPa, while in the design of 1 mm in resolution and 75 μm in thickness, the maximal pressure can be extend up to 200 kPa. When the applied pressure goes beyond the measurement range, the response could become highly non-linear and saturated. Overall, the spatial resolution and the membrane thickness of the IMA could be the determinant factors in the sensor performance (i.e., device sensitivity and dynamic range), allowing highly customizable sensors for a wide range of specifications and applications.

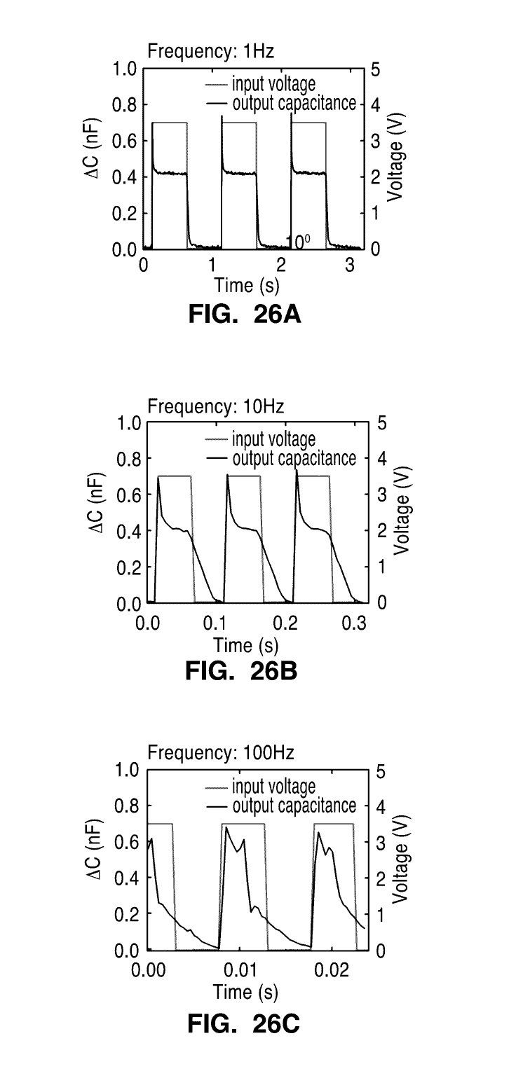

Experiments were conducted to characterize the response time of the IMA devices. A pulsed contact pressure (of ˜1.4 kPa) in the frequency ranging from 1 Hz to 100 Hz has been applied to the device surface through an electromagnetically driven pin actuator. Both the driving voltages to the actuator and the capacitive changes are recorded. As shown in FIG. 26A through FIG. 26C, the capacitive changes of the sensor repeats in the same frequency to the corresponding voltages applied to the pin actuator, suggesting that the sensor can response to the pressure in the frequency up to 100 Hz. It is worth noting that the distortion of the recorded pressure signals likely attributes to the open-loop operation of the load applied by the actuator (i.e., the rapid rise edge and slow recovery phase of the capacitive readings).

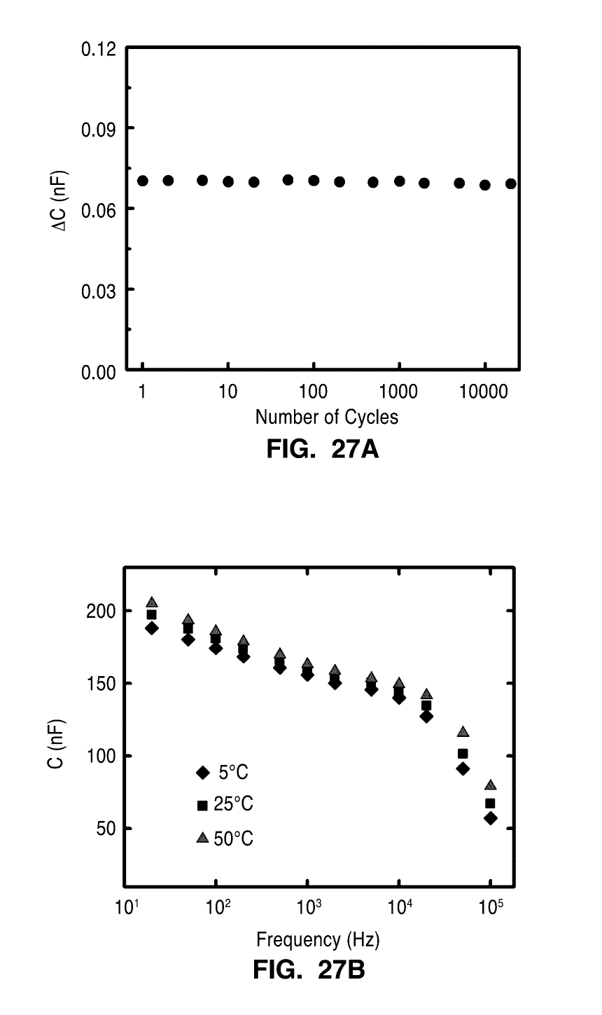

To investigate the mechanical reliability and robustness of the IMA devices, repeatability tests are conducted by recording the capacitive changes of a single sensing element as a function of press-and-release cycles. As shown in FIG. 27A, the sensor maintains the relatively constant capacitive changes of less than 3% variation even after 20,000 cycles of pressure/force loads, illustrating the mechanical robustness and reliability of the IMA sensing devices. Moreover, the environmental thermal influence on the device electrical performance has been investigated. In principle, both the unit—are EDL capacitance and the volume of the IL droplets can be affected by the temperature, resulting in the changes of overall device capacitance.

FIG. 27B illustrates the frequency spectrums of the initial capacitance with the liquid-solid contact area of 4.2 mm² over different temperatures from 5° C. to 50° C. As can be seen, the temperature variation has posed a minor impact on the interfacial capacitance, i.e., less than 10% increase over the tested temperature range. In addition, the minimal capacitive change over the temperature fluctuation has been observed at the frequency of 1 kHz (a 4% change in total), which could be taken into a consideration in the IMA operation. In addition, any bending or stretching could likely result in a change in the initial capacitance as the flexible membranes and the droplets underneath are mechanically deformed. In such a case, we need to adjust the sensing curve with a new initial setting (i.e., initial capacitance) for the subsequent pressure and force measurement.

To demonstrate the utility of the iontronic devices, we have applied the IMA sensors to resolve the surface topology and to record dynamic blood pulses. The two IMA devices have been devised to map the static surface topology, 6×6 and 12×12 arrays with the pixel resolutions of 1.5 mm and 2.0 mm, respectively. By placing a polymeric stamp and a weight (of 363 g) on the top of the surface, the capacitance value of each sensing unit can be scanned and processed by a readout circuitry (see FIG. 23).

Surface topology measurements were made with corresponding stamps made of PDMS elastomer, from which the pressure distribution was clearly resolved. The accurate mapping of the spatial pressure distribution is highly relied on the large EDL capacitance. Unlike the capacitance in classic solid-state capacitive sensors, the novel interfacial capacitance in each sensing element is on the order of a few nF, allowing to largely reduce the interference from the environments, e.g., stray capacitance and electrical field from the neighbor sensing units. Although the pixel resolution is currently limited by the printed droplet size, the further improvement on microdroplet dispensing can obtain a higher sensor resolution.

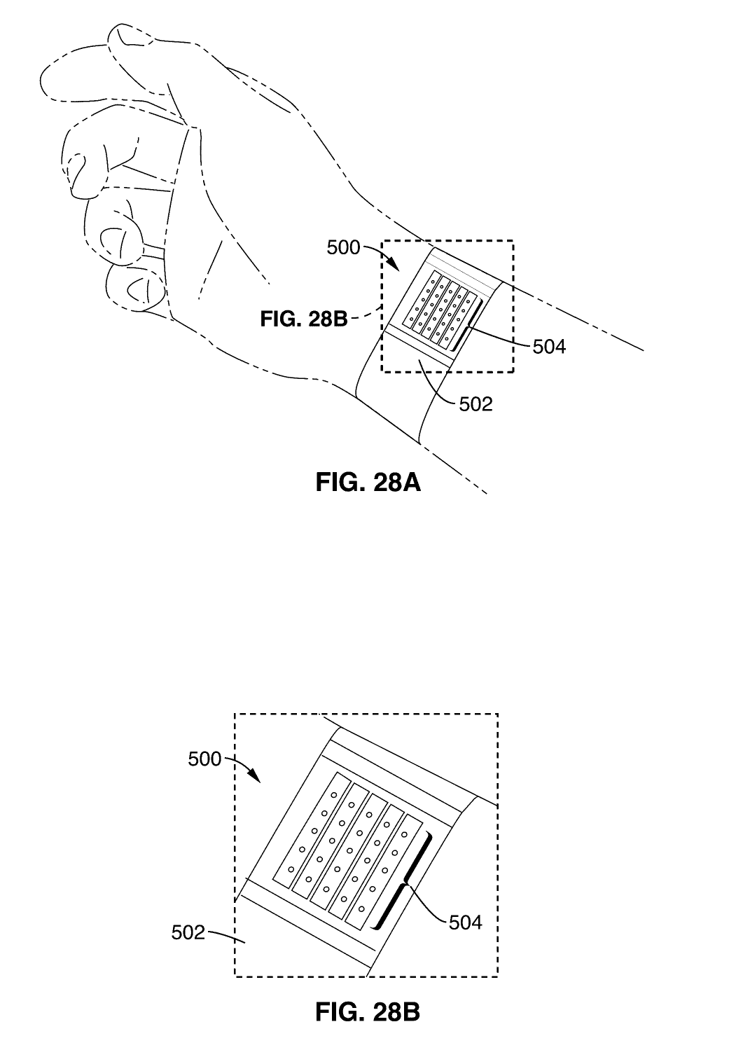

To further extend the flexibility and adaptability of the IMA devices to artificial tactile applications, a sensor array was configured to detect fine surface topology, such as Braille letters. The custom IMA comprised of 2×3 pixels with the spatial resolution of 2.3 mm (to match with the standard Braille letters), and was worn in a fingertip set up instead of the wrist setup of FIG. 28A and FIG. 28B. For the fingertip reading of Braille texts, a gentle contact pressure has been applied to the text surface by the finger. The raised dotted impressions of each Braille character cause the membrane deformation in the corresponding droplet sensing units, which can be subsequently detected by the changes of the interfacial capacitance. Using the finger-mounted IMA device, the letter of “BRAILLE” has been successfully resolved, in which each pressure reading is converted to a digital colorimetric scale. Digital recording of the tactile sensing can be further processed and transmitted into audible readings, and thus, it can be of potential use for Braille education for visually impaired patients.

Referring to FIG. 28A to FIG. 31, the ultrahigh device sensitivity and rapid response time of the iontronic droplet sensors of the present description were implemented in non-invasive cardiovascular pressure recording. As shown in FIGS. 28A and 28B, a wristband IMA device 500 comprised of a 5×5 sensor array 504 with the spatial resolution of 3 mm was positioned in contact with the skin above the radial artery and fixed by a plastic wristband 502.

Real-time pulse recording was performed by scanning all the sensing elements covering the skin area of 15×15 mm² at the sampling frequency of 1 kHz in each unit. The IMA device 500 enables two important functions in the pulse recording. First of all, the sensor spatially maps the pulse on the skin surface, from which the sites of the maximal pressure variations can be located.

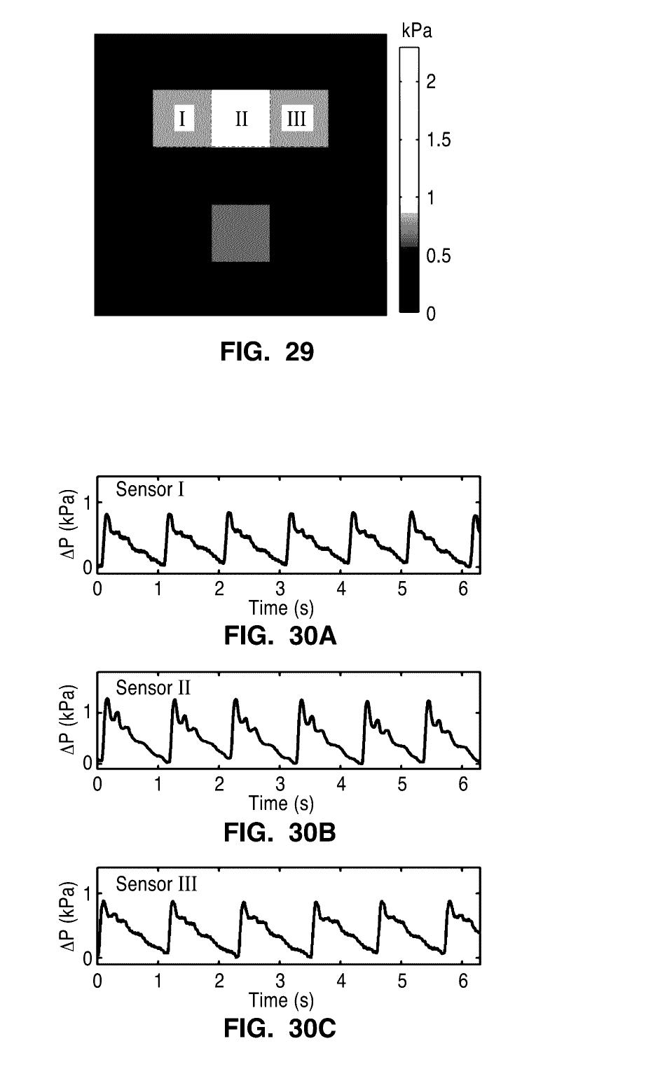

Comparing the pressure mapping results in FIG. 29 with the sensor position shown in FIG. 28A and FIG. 28B, the pressure sensing units right above the radial artery provide the highest capacitive recordings (marked as I, II, III) as expected, and thus, closely reflects the cardiovascular pressure readings using the tonometry principle.

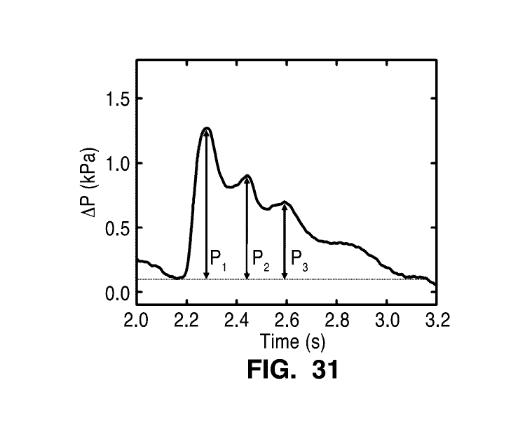

In the following step, the pressure wave forms were continuously tracked from these optimal sensing positions. FIG. 30A through FIG. 30C shows the continuous pulse recordings from the three pixels, respectively. As can be seen, the maximal pulse variation is around 1.2 kPa recorded by sensor II. FIG. 31 provides a close-up analysis of a single cardiac cycle, which has been characterized into three peaks (P1, P2 and P3). These maxima are caused by traveling waves of the systolic phase and diastolic phase of the blood pressure conducted in the elastic cardiovascular vessels. Clinically significant parameters, such as the radial augmentation index, AI, (=P1/P2) and the reflection index, RI, (=P1/P3), can be directly extracted and computed from the maximal pulse recordings, which can be potentially used to screen the arterial compliance and arteriolar tone. Moreover, the radial pulse waveforms recorded at the optimal sites can be further processed to estimate the central aortic pressure and cardiac output, which reflects the important cardiovascular events and the health states. Though a similar measurement has been conducted recently through a single-channel capacitive sensor, the IMA offers the combined advantage of simultaneous pressure mapping of the optimal recording area and continuous tracking of the blood pressure waveform, in addition to its flexible transparent packaging. In this fashion, the IMA can serve as a flexible sensing device that is highly attractive for the emerging wearable health monitoring applications, in comparison with the conventional invasive cardiovascular monitoring.

In other embodiments (not shown), the liquid-based pressure sensor can be developed into a wireless LC pressure sensor, which can be used to monitoring the pressure inside of human body. For example, the capacitive pressure sensor can be embedded in a guidewire for blood pressure monitoring.

It is appreciated that the sensing device sensitivity and dynamic range disclosed herein can be highly customizable based on various design parameters, e.g., spatial resolution, membrane thickness and chamber height, and therefore, it can be readily configured for various pressure sensing applications. In addition, the fluidic nature of the sensors enables rapid mechanical responses (on the order of a few milliseconds). Moreover, the IMA sensor exhibits high repeatability (less than 3% variation in capacitive readings) over more than 20,000 cycles of external loads. In addition, the simple device is optically transparent and can be massive produced with high reliability yet low cost.

The microdroplet sensors disclosed herein enable a highly transformative platform of tactile sensing for a wide range of emerging applications, including robotics, medical prosthetics, surgical instruments, video gaming and wearable computing.

From the description herein, it will be appreciated that that the present disclosure encompasses multiple embodiments which include, but are not limited to, the following:

1. An array of droplet-based sensors, comprising: a plurality of sensing chambers each having interior volumes housed by a first substrate and a second substrate; wherein each of the plurality of sensing chambers comprise a substrate separation structure maintaining a periphery of said first and second substrates at a fixed separation distance to form each of the plurality of sensing chambers; at least a first electrode and second electrode coupled to the interior volume of each of the plurality of sensing chambers; and an electrolytic liquid retained in each of said plurality of sensing chambers; said electrolytic liquid disposed in the sensing chamber to form a contact with said first and second electrodes; wherein in response to an applied force, at least one of said substrates deforms, thereby changing the contact between the electrolytic liquid and the first and second electrodes and thus the electrical properties between said first and second electrodes.

2. The array of any preceding embodiment, wherein said properties are selected from the group of electrical properties consisting of interfacial electric double layers (EDL) capacitance, resistance, impedance including both resistance and capacitance, and inductance which are sensed as a measure of pressure/force applied to each of said droplet-based pressure/force sensors.

3. The array of any preceding embodiment, wherein the electrolytic liquid comprises an electrolyte droplet.

4. The array of any preceding embodiment, wherein the electrolytic liquid comprises a column of electrolyte.

5. The array of any preceding embodiment, wherein the electrolytic liquid fills the sensing chamber.

6. The array of any preceding embodiment, wherein the electrolytic liquid is centrally aligned in the sensing chamber.

7. The array of any preceding embodiment, wherein the electrolytic liquid is aligned to one side of the sensing chamber.

8. The array of any preceding embodiment, wherein one or more surfaces of the chamber comprise a hydrophobic region to retain the electrolytic liquid at a specified location within the chamber.

9. The array of any preceding embodiment, wherein one or more surfaces of the chamber comprise a micropillar structure to retain the electrolytic liquid at a specified location within the chamber.

10. The array of any preceding embodiment, further comprising: a channel in fluid communication with the chamber; said first electrode and second electrode coupled to one or more surfaces of the channel; wherein the electrolytic liquid is forced into the channel in response to the applied force to the sensor.

11. The array of any preceding embodiment, further comprising: a second channel in fluid communication with the chamber; said first electrode and second electrode coupled to one or more surfaces of the second channel; wherein the electrolytic liquid is forced into the second channel in response to the applied force to the sensor.

12. The array of any preceding embodiment, wherein the force comprises a normal, shear or pressure force applied to the sensor.

13. The array of any preceding embodiment, wherein at least one of said first electrode and said second electrode is connected in common within each of said array of sensors.

14. The array of any preceding embodiment, wherein both said first electrode and said second electrode are connected in common within each of said array of droplet-based pressure/force sensors.

15. The array of any preceding embodiment, wherein said first electrode and said second electrode are disposed on opposing sides of the sensor chamber.

16. The array of any preceding embodiment, wherein said first electrode and said second electrode are disposed in a coplanar orientation on one side of the sensor chamber.

17. The array of any preceding embodiment, wherein one or more of the first and second substrates comprises a cavity to form said separation structure.

18. The array of any preceding embodiment, wherein at least one of said first or second substrates is flexible.

19. A liquid-based sensing apparatus, comprising: at least one sensing chamber comprising an interior volume housed by a first substrate and a second substrate; wherein the sensing chamber comprises a substrate separation structure maintaining a periphery of said first and second substrates at a fixed separation distance to form the sensing chamber; at least a first electrode and second electrode coupled to the interior volume of the sensing chamber; and an electrolytic liquid retained in the sensing chamber; said electrolytic liquid disposed in the sensing chamber to form a contact with said first and second electrodes; wherein in response to an applied force, at least one of said substrates deforms, thereby changing the contact between the electrolytic liquid and the first and second electrodes and thus the electrical properties between said first and second electrodes.

20. The apparatus of any preceding embodiment, wherein said properties are selected from the group of electrical properties consisting of interfacial electric double layers (EDL) capacitance, resistance, impedance including both resistance and capacitance, and inductance which are sensed as a measure of pressure/force applied to each of said droplet-based pressure/force sensors.

21. The apparatus of any preceding embodiment, wherein the electrolytic liquid comprises an electrolyte droplet.

22. The apparatus of any preceding embodiment, wherein the electrolytic liquid comprises a column of electrolyte.

23. The apparatus of any preceding embodiment, wherein the electrolytic liquid fills the sensing chamber.

24. The apparatus of any preceding embodiment, wherein the electrolytic liquid is centrally aligned in the sensing chamber.

25. The apparatus of any preceding embodiment, wherein the electrolytic liquid is aligned to one side of the sensing chamber.

26. The apparatus of any preceding embodiment, wherein one or more surfaces of the chamber comprise a hydrophobic region to retain the electrolytic liquid at a specified location within the chamber.

27. The apparatus of any preceding embodiment, wherein one or more surfaces of the chamber comprise a micropillar structure to retain the electrolytic liquid at a specified location within the chamber.

28. The apparatus of any preceding embodiment, further comprising: a channel in fluid communication with the chamber; said first electrode and second electrode coupled to one or more surfaces of the channel; wherein the electrolytic liquid is forced into the channel in response to the applied force to the sensor.

29. The apparatus of any preceding embodiment, further comprising: a second channel in fluid communication with the chamber; a third electrode and fourth electrode coupled to one or more surfaces of the second channel; wherein the electrolytic liquid is forced into the second channel in response to the applied force to the sensor; and wherein the first electrode, second electrode, third electrode and fourth electrode are capable of individually detecting the electrical properties of the first channel and the second channel.

30. The apparatus of any preceding embodiment, wherein the force comprises a normal, shear or pressure force applied to the sensor.

31. The apparatus of any preceding embodiment, wherein said first electrode and said second electrode are disposed on opposing sides of the sensor chamber.

32. The apparatus of any preceding embodiment, wherein said first electrode and said second electrode are disposed in a coplanar orientation on one side of the sensor chamber.

33. The apparatus of any preceding embodiment, wherein one or more of the first and second substrates comprises a cavity to form said separation structure.

34. The apparatus of any preceding embodiment, wherein at least one of said first or second substrates is flexible.

35. The apparatus of any preceding embodiment, wherein said first and second electrodes are configured in proximal configurations selected from a group of configurations consisting of co-planar electrodes, interdigitated electrodes, spiral forms, and combinations thereof.

36. The apparatus of any preceding embodiment, wherein said first and second electrodes are disposed in an interdigitated configuration.

37. The apparatus of any preceding embodiment, wherein said first and second electrodes are disposed proximal one another in an elongate pattern.

38. The apparatus of any preceding embodiment, wherein the elongate pattern comprises a spiral pattern having curving or straight line segments in a polygonal pattern, or a combination of curving and straight line segments in a polygonal pattern.

39. A droplet-based pressure/force sensor apparatus, comprising: at least one sensing chamber within an interior volume of a housing having a first and a second substrate between which are disposed a substrate separation structure maintaining a periphery of said first and second substrates at a fixed separation distance to form said sensing chamber; wherein at least one of said first or second substrates is flexible; at least a first electrode and second electrode extending into the interior volume of said housing; and an electrolyte droplet retained to partially fill said sensing chamber and be in contact with, or disposed for contact with, said first and second electrodes; wherein said electrolyte droplet is in contact with a portion of said substrate separation structure, whereby in response to deformation of said first or second substrate expands away from that portion of said substrate separation structure to which it is in contact; wherein in response to an applied force, at least one of said substrates deforms, changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to said apparatus.

40. The apparatus of any preceding embodiment, further comprising: a third electrode and fourth electrode extending into the interior volume of said housing; and wherein the third electrode and fourth electrodes are configured to sense electrical properties within said chamber from the first and second electrodes.

41. A droplet-based pressure/force sensor apparatus, comprising: at least one sensing chamber within an interior volume of a housing having a first and a second substrate between which are disposed a substrate separation structure maintaining a periphery of said first and second substrates at a fixed separation distance to form said sensing chamber; wherein at least one of said first or second substrates is flexible; at least a first electrode and second electrode are disposed on an interior surface of said first substrate, said second substrate, or both said first substrate and said second substrate, into the interior volume of said housing, and in separation from one another; and an electrolyte droplet retained in said sensing chamber disposed for contact with said first and second electrodes; wherein in response to pressure or force at least one of said substrates which is flexible, deforms changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to said apparatus.

42. The apparatus of any preceding embodiment, further comprising: a third electrode and fourth electrode disposed on an interior surface of said first substrate, said second substrate, or both said first substrate and said second substrate, into the interior volume of said housing, and in separation from one another; and wherein the third electrode and fourth electrodes are configured to sense electrical properties within said chamber from the first and second electrodes.

43. A droplet-based pressure/force sensor apparatus, comprising: at least one sensing chamber within an interior volume of a housing having a first and a second substrate between which are disposed a substrate separation structure maintaining a periphery of said first and second substrates at a fixed separation distance to form said sensing chamber; wherein said sensing chamber comprises at least one portion having a reduced cross-sectional area; wherein at least one of said first or second substrates is flexible; at least a first electrode and second electrode are disposed on an interior surface of said first substrate, said second substrate, or both said first substrate and said second substrate, into the interior volume of said housing, and in separation from one another; and an electrolyte droplet retained in said sensing chamber disposed for contact with said first and second electrodes and configured for expansion through said portion of said sensing chamber having a reduced cross-sectional area; wherein in response to an applied pressure or force, at least one of said substrates which is flexible, deforms changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to said apparatus.

44. The apparatus of any preceding embodiment, wherein said sensing chamber has multiple portions with reduced cross-sectional area.

45. The apparatus of any preceding embodiment, wherein at least one of the electrodes in said sensing chamber do not make contact with said electrolyte droplet until a threshold level of pressure/force is applied to said apparatus.

46. An array of droplet-based pressure/force sensors, comprising: at plurality of sensing chambers having interior volumes housed by a first substrate and a second substrate, between which are disposed a substrate separation structure maintaining a periphery of said first and second substrates at a fixed separation distance to form each of said plurality of sensing chambers; wherein at least one of said first or second substrates is flexible; at least a first electrode and second electrode extending into the interior volume of said housing; and an electrolyte droplet retained in said sensing chamber disposed for contact with said first and second electrodes; wherein in response to applied normal/shear pressure/force at least one of said substrates which is flexible, deforms changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to each of said droplet-based pressure/force sensors.

47. The array of any preceding embodiment, wherein at least one of said first electrode and said second electrode is connected in common within each of said array of droplet-based pressure/force sensors.

48. The array of any preceding embodiment, wherein both said first electrode and said second electrode are connected in common within each of said array of droplet-based pressure/force sensors.

49. A droplet-based pressure/force sensor apparatus, comprising: at least one sensing chamber within an interior volume of a housing having a first and a second substrate between which are disposed a substrate separation structure maintaining a periphery of said first and second substrates at a fixed separation distance to form said sensing chamber; wherein at least one of said first or second substrates is flexible; at least a first electrode and second electrode extending into the interior volume of said housing; and an electrolyte droplet retained to fill said sensing chamber in contact with said first and second electrodes; wherein in response to applied normal/shear pressure/force at least one of said substrates which is flexible, deforms changing the resistance between said first electrode and said second electrode as a measure of pressure/force applied to said apparatus.

50. A droplet-based pressure/force sensor apparatus, comprising: at least one sensing chamber within an interior volume of a housing having a first and a second substrate between which are disposed a substrate separation structure maintaining a periphery of said first and second substrates at a fixed separation distance to form said sensing chamber; wherein at least one of said first or second substrates is flexible; at least a first electrode and second electrode extending into the interior volume of said housing; and an electrolyte droplet retained to partially fill said sensing chamber and be in contact with, or disposed for contact with, said first and second electrodes; wherein said electrolyte droplet is in contact with a portion of said substrate separation structure, whereby in response to deformation of said first or second substrate expands away from that portion of said substrate separation structure to which it is in contact; wherein in response to applied normal/shear pressure/force at least one of said substrates which is flexible, deforms changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to said apparatus.

51. A droplet-based pressure/force sensor apparatus, comprising: at least one sensing chamber within an interior volume of a housing having a first and a second substrate between which are disposed a substrate separation structure maintaining a periphery of said first and second substrates at a fixed separation distance to form said sensing chamber; wherein at least one of said first or second substrates is flexible; at least a first electrode and second electrode are disposed on an interior surface of said first substrate, said second substrate, or both said first substrate and said second substrate, into the interior volume of said housing, and in separation from one another; and an electrolyte droplet retained in said sensing chamber disposed for contact with said first and second electrodes; wherein in response to applied normal/shear pressure/force at least one of said substrates which is flexible, deforms changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to said apparatus.

52. The apparatus of any preceding embodiment, wherein a first electrode and second electrode are disposed on either said first substrate or said second substrate.

53. The apparatus of any preceding embodiment, wherein at least one of said first electrode and said second electrode are not disposed in contact with said droplet at a first applied level of pressure/force, and only establish contact at a second level of pressure/force.

54. The apparatus of any preceding embodiment, wherein establishing of said contact creates a switch mechanism which activates at a desired pressure/force threshold.

55. The apparatus of any preceding embodiment, wherein a first electrode and second electrode are disposed on said first substrate, and a third electrode and a fourth electrode are disposed on said second substrate.

56. The apparatus of any preceding embodiment, wherein said first electrode or second electrode is in common connection with said third electrode or said fourth electrode.

57. The apparatus of any preceding embodiment, wherein at least one of said first electrode, said second electrode, said third electrode, or said fourth electrode are not in contact with said droplet at a first applied level of pressure/force, and only establishes contact at a second level of pressure/force.

58. The apparatus of any preceding embodiment, wherein establishing of said contact creates a switch mechanism which activates at a desired pressure/force threshold.

59. The apparatus of any preceding embodiment, wherein said first electrode and said second electrode have a different separation distance, than that between said third electrodes and said fourth electrode, providing different pressure/force sensing profiles.

60. The apparatus of any preceding embodiment, wherein said first and second electrodes can be configured in proximal configurations selected from a group of configurations consisting of co-planar electrodes, interdigitated electrodes, spiral forms, and combinations thereof which can each have any desired shape.

61. A droplet-based pressure/force sensor apparatus, comprising: at least one sensing chamber within an interior volume of a housing having a first and a second substrate between which are disposed a substrate separation structure maintaining a periphery of said first and second substrates at a fixed separation distance to form said sensing chamber; wherein said sensing chamber is configured at least one portion having a reduced cross-sectional area; wherein at least one of said first or second substrates is flexible; at least a first electrode and second electrode are disposed on an interior surface of said first substrate, said second substrate, or both said first substrate and said second substrate, into the interior volume of said housing, and in separation from one another; and an electrolyte droplet retained in said sensing chamber disposed for contact with said first and second electrodes and configured for expansion through said portion of said sensing chamber having a reduced cross-sectional area; wherein in response to applied normal/shear pressure/force at least one of said substrates which is flexible, deforms changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to said apparatus.

62. The apparatus of any preceding embodiment, wherein said sensing chamber has multiple portions with reduced cross-sectional area.

63. The apparatus of any preceding embodiment, wherein at least one of the electrodes in said sensing chamber do not make contact with said electrolyte droplet until a threshold level of pressure/force is applied to said apparatus.

64. A droplet-based pressure/force sensor apparatus, comprising: a first sensing chamber within an interior volume of a first housing having a first and a second substrate between which are disposed a first substrate separation structure maintaining a periphery of said first and second substrates at a fixed separation distance to form said first sensing chamber; a second sensing chamber within an interior volume of a second housing having a third and fourth substrate between which are disposed a second substrate separation structure maintaining a periphery of said third and fourth substrates at a fixed separation distance to form said second sensing chamber; wherein said first sensing chamber and said second sensing chamber are disposed proximal one another; wherein at least one of said first, second, third or fourth substrates is flexible; at least a first electrode and second electrode are disposed on an interior surface of said first substrate, said second substrate, or both said first substrate and said second substrate, into the interior volume of said first housing, and in separation from one another; at least a third electrode and fourth electrode are disposed on an interior surface of said third substrate, fourth second substrate, or both said third substrate and said fourth substrate, into the interior volume of said second housing, and in separation from one another; and a first electrolyte droplet retained in said first sensing chamber disposed for contact with said first and second electrodes and a second electrolyte droplet retained in said second sensing chamber disposed for contact with said third and fourth electrodes; wherein in response to applied deformation normal/shear pressure/force upon said first housing and/or said second housing electrical properties change between said first and second electrodes, and third and fourth electrodes, and between the set of first and second electrodes and the third and fourth electrodes, said properties selected from the group of electrical properties consisting of interfacial electric double layers (EDL) capacitance, resistance, impedance including both resistance and capacitance, and inductance which are sensed as a measure of pressure/force applied to said apparatus.

65. The apparatus of any preceding embodiment, wherein said first and second sense chambers are disposed in a interdigitated configuration.

66. The apparatus of any preceding embodiment, wherein said first and second sense chambers are disposed proximal one another in an elongate pattern.

67. The apparatus of any preceding embodiment, wherein the elongate pattern comprises a spiral pattern having curving or straight line segments in a polygonal pattern, or a combination of curving and straight line segments in a polygonal pattern.

68. A droplet-based pressure/force sensor apparatus, comprising: at least one sensing chamber within an interior volume of a housing having a first and a second substrate between which are disposed a substrate separation structure maintaining a periphery of said first and second substrates at a fixed separation distance to form said sensing chamber; wherein at least one of said first or second substrates is flexible; at least a first electrode and second electrode are disposed into the interior volume of said housing, and in separation from one another; at least one electrode separation structure disposed within said housing for preventing said first electrode and said second electrode from coming into sufficiently close proximity to create an undesired low level of resistance between said first and second electrodes; and an electrolyte droplet retained in said sensing chamber disposed for contact with said first and second electrodes; wherein in response to applied normal/shear pressure/force at least one of said substrates which is flexible, deforms changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to said apparatus.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An array of droplet-based sensors, comprising:
a plurality of sensing chambers each having interior volumes housed by a first substrate and a second substrate;
wherein each of the plurality of sensing chambers comprise a substrate separation structure maintaining a periphery of said first and second substrates at a fixed separation distance to form each of said plurality of sensing chambers;
at least a first electrode and second electrode coupled to the interior volume of each of the plurality of sensing chambers; and
an electrolytic liquid retained in each of the plurality of sensing chambers;
said electrolytic liquid disposed in the sensing chamber to form a contact with said first and second electrodes;
wherein in response to an applied force, at least one of said substrates deforms, thereby changing the contact between the electrolytic liquid and the first and second electrodes and thus the electrical properties between said first and second electrodes.

2. The array of claim 1, wherein said properties are selected from the group of electrical properties consisting of interfacial electric double layers (EDL) capacitance, resistance, impedance including both resistance and capacitance, and inductance which are sensed as a measure of pressure/force applied to each of said droplet-based pressure/force sensors.

3. The array of claim 1, wherein the electrolytic liquid comprises an electrolyte droplet.

4. The array of claim 1, wherein the electrolytic liquid comprises a column of electrolyte.

5. The array of claim 1, wherein the electrolytic liquid fills the sensing chamber.

6. The array of claim 1, wherein the electrolytic liquid is centrally aligned in the sensing chamber.

7. The array of claim 1, wherein the electrolytic liquid is aligned to one side of the sensing chamber.

8. The array of claim 1, wherein one or more surfaces of the chamber comprise a hydrophobic region to retain the electrolytic liquid at a specified location within the chamber.

9. The array of claim 1, wherein one or more surfaces of the chamber comprise a micropillar structure to retain the electrolytic liquid at a specified location within the chamber.

10. The array of claim 1, further comprising:
a channel in fluid communication with the chamber;
said first electrode and second electrode coupled to one or more surfaces of the channel;
wherein the electrolytic liquid is forced into the channel in response to the applied force to the sensor.

11. The array of claim 10, further comprising:
a second channel in fluid communication with the chamber;
said first electrode and second electrode coupled to one or more surfaces of the second channel;
wherein the electrolytic liquid is forced into the second channel in response to the applied force to the sensor.

12. The array of claim 1, wherein the force comprises a normal, shear or pressure force applied to the sensor.

13. The array of claim 1, wherein at least one of said first electrode and said second electrode is connected in common within each of said array of sensors.

14. The array of claim 1, wherein both said first electrode and said second electrode are connected in common within each of said array of droplet-based pressure/force sensors.

15. The array of claim 1, wherein said first electrode and said second electrode are disposed on opposing sides of the sensor chamber.

16. The array of claim 1, wherein said first electrode and said second electrode are disposed in a coplanar orientation on one side of the sensor chamber.

17. The array of claim 1, wherein one or more of the first and second substrates comprises a cavity to form said separation structure.

18. The array of claim 1, wherein at least one of said first or second substrates is flexible.

19. A liquid-based sensing apparatus, comprising:
at least one sensing chamber comprising an interior volume housed by a first substrate and a second substrate;
wherein the sensing chamber comprises a substrate separation structure maintaining a periphery of said first and second substrates at a fixed separation distance to form the sensing chamber;
at least a first electrode and second electrode coupled to the interior volume of the sensing chamber; and
an electrolytic liquid retained in the sensing chamber;
said electrolytic liquid disposed in the sensing chamber to form a contact with said first and second electrodes;
wherein in response to an applied force, at least one of said substrates deforms, thereby changing the contact between the electrolytic liquid and the first and second electrodes and thus the electrical properties between said first and second electrodes.

20. The apparatus of claim 19, wherein said properties are selected from the group of electrical properties consisting of interfacial electric double layers (EDL) capacitance, resistance, impedance including both resistance and capacitance, and inductance which are sensed as a measure of pressure/force applied to each of said droplet-based pressure/force sensors.

21. The apparatus of claim 19, wherein the electrolytic liquid comprises an electrolyte droplet.

22. The apparatus of claim 19, wherein the electrolytic liquid comprises a column of electrolyte.

23. The apparatus of claim 19, wherein the electrolytic liquid fills the sensing chamber.

24. The apparatus of claim 19, wherein the electrolytic liquid is centrally aligned in the sensing chamber.

25. The apparatus of claim 19, wherein the electrolytic liquid is aligned to one side of the sensing chamber.

26. The apparatus of claim 19, wherein one or more surfaces of the chamber comprise a hydrophobic region to retain the electrolytic liquid at a specified location within the chamber.

27. The apparatus of claim 19, wherein one or more surfaces of the chamber comprise a micropillar structure to retain the electrolytic liquid at a specified location within the chamber.

28. The apparatus of claim 19, further comprising:
a channel in fluid communication with the chamber;
said first electrode and second electrode coupled to one or more surfaces of the channel;
wherein the electrolytic liquid is forced into the channel in response to the applied force to the sensor.

29. The apparatus of claim 28, further comprising:
a second channel in fluid communication with the chamber;
a third electrode and fourth electrode coupled to one or more surfaces of the second channel;
wherein the electrolytic liquid is forced into the second channel in response to the applied force to the sensor; and
wherein the first electrode, second electrode, third electrode and fourth electrode are capable of individually detecting the electrical properties of the first channel and the second channel.

30. The apparatus of claim 19, wherein the force comprises a normal, shear or pressure force applied to the sensor.

31. The apparatus of claim 19, wherein said first electrode and said second electrode are disposed on opposing sides of the sensor chamber.

32. The apparatus of claim 19, wherein said first electrode and said second electrode are disposed in a coplanar orientation on one side of the sensor chamber.

33. The apparatus of claim 19, wherein one or more of the first and second substrates comprises a cavity to form said separation structure.

34. The apparatus of claim 19, wherein at least one of said first or second substrates is flexible.

35. The apparatus of claim 19, wherein said first and second electrodes are configured in proximal configurations selected from a group of configurations consisting of co-planar electrodes, interdigitated electrodes, spiral forms, and combinations thereof.

36. The apparatus as recited in claim 19, wherein said first and second electrodes are disposed in an interdigitated configuration.

37. The apparatus as recited in claim 19, wherein said first and second electrodes are disposed proximal one another in an elongate pattern.

38. The apparatus as recited in claim 37, wherein the elongate pattern comprises a spiral pattern having curving or straight line segments in a polygonal pattern, or a combination of curving and straight line segments in a polygonal pattern.

39. A droplet-based pressure/force sensor apparatus, comprising:
at least one sensing chamber within an interior volume of a housing having a first and a second substrate between which are disposed a substrate separation structure maintaining a periphery of said first and second substrates at a fixed separation distance to form said sensing chamber;
wherein at least one of said first or second substrates is flexible;
at least a first electrode and second electrode extending into the interior volume of said housing; and
an electrolyte droplet retained to partially fill said sensing chamber and be in contact with, or disposed for contact with, said first and second electrodes;
wherein said electrolyte droplet is in contact with a portion of said substrate separation structure, whereby in response to deformation of said first or second substrate expands away from that portion of said substrate separation structure to which it is in contact;
wherein in response to an applied force, at least one of said substrates deforms, changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to said apparatus.

40. The apparatus of claim 39, further comprising:
a third electrode and fourth electrode extending into the interior volume of said housing; and
wherein the third electrode and fourth electrodes are configured to sense electrical properties within said chamber from the first and second electrodes.

41. A droplet-based pressure/force sensor apparatus, comprising:
at least one sensing chamber within an interior volume of a housing having a first and a second substrate between which are disposed a substrate separation structure maintaining a periphery of said first and second substrates at a fixed separation distance to form said sensing chamber;
wherein at least one of said first or second substrates is flexible;
at least a first electrode and second electrode are disposed on an interior surface of said first substrate, said second substrate, or both said first substrate and said second substrate, into the interior volume of said housing, and in separation from one another; and
an electrolyte droplet retained in said sensing chamber disposed for contact with said first and second electrodes;
wherein in response to pressure or force at least one of said substrates which is flexible, deforms changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to said apparatus.

42. The apparatus of claim 41, further comprising:
a third electrode and fourth electrode disposed on an interior surface of said first substrate, said second substrate, or both said first substrate and said second substrate, into the interior volume of said housing, and in separation from one another; and
wherein the third electrode and fourth electrodes are configured to sense electrical properties within said chamber from the first and second electrodes.

43. A droplet-based pressure/force sensor apparatus, comprising:
at least one sensing chamber within an interior volume of a housing having a first and a second substrate between which are disposed a substrate separation structure maintaining a periphery of said first and second substrates at a fixed separation distance to form said sensing chamber;
wherein said sensing chamber comprises at least one portion having a reduced cross-sectional area;
wherein at least one of said first or second substrates is flexible;
at least a first electrode and second electrode are disposed on an interior surface of said first substrate, said second substrate, or both said first substrate and said second substrate, into the interior volume of said housing, and in separation from one another; and
an electrolyte droplet retained in said sensing chamber disposed for contact with said first and second electrodes and configured for expansion through said portion of said sensing chamber having a reduced cross-sectional area;
wherein in response to an applied pressure or force, at least one of said substrates which is flexible, deforms changing electrical properties between said first and second electrodes, said properties selected from the group of electrical properties consisting of interfacial electric double layers (EDL) capacitance, resistance, impedance including both resistance and capacitance, and inductance which are sensed as a measure of pressure/force applied to said apparatus.

44. The apparatus of claim 43, wherein said sensing chamber has multiple portions with reduced cross-sectional area.

45. The apparatus of claim 43, wherein at least one of the electrodes in said sensing chamber do not make contact with said electrolyte droplet until a threshold level of pressure/force is applied to said apparatus.
